(19) United States
(12) Patent Application Publication
(10) Pub. No.: US 2016/0303363 A1
(43) Pub. Date: Oct. 20, 2016

(54) NMES Garment

(71) Applicants: Michael P. GIROUARD, Clemens, NC (US); Jonie M. GIROUARD, Clemens, NC (US)

(72) Inventors: Michael P. GIROUARD, Clemens, NC (US); Jonie M. GIROUARD, Clemens, NC (US)

(21) Appl. No.: 15/073,580

(22) Filed: Mar. 17, 2016

Related U.S. Application Data
(60) Provisional application No. 62/309,269, filed on Mar. 16, 2016.
(60) Provisional application No. 62/134,407, filed on Mar. 17, 2015.

(51) Int. Cl. A61N 1/04; A61N 1/36

(52) U.S. Cl. CPC A61N 1/0484; A61N 1/0452; A61N 1/0456; A61N 1/36003; A61N 1/36014

(57) ABSTRACT

The present invention relates to apparatuses, methods, and systems for simulating low and/or high intensity exercise. More particularly, the present invention relates to an exercise mimetic device for simulating low and/or high intensity exercise using low intensity electrical stimulation to generate low intensity muscle contractions such as a wearable garment that preferably imitates exercise by eliciting low grade muscle contractions in several of the larger skeletal muscle groups in the body. The apparatus of various embodiments of the present invention is a neuromuscular electrostimulation (NMES) device/garment with a control unit that is wirelessly connected to and controls a stimulator unit that generates and transmits a low intensity electrical stimulation within certain unique parameters. In various embodiments, the NMES device/garment is for treating conditions including but not limited to obesity, obesity related conditions such as diabetes, muscle toning, and/or other conditions benefitted by exercise. In various embodiments, the NMES device/garment is an over the counter (OTC) NMES device/garment.

### CROSS-REFERENCE TO RELATED APPLICATIONS

Incorporated herein by reference is U.S. Provisional Patent Application No. 62/134,407, filed 17 Mar. 2015, and U.S. Provisional Patent Application No. 62/309,269, filed 16 Mar. 2016, priority of which are hereby claimed.

### STATEMENT REGARDING FEDERALLY SPONSORED RESEARCH OR DEVELOPMENT

Not applicable

### REFERENCE TO A “MICROFICHE APPENDIX” Not applicable

### BACKGROUND OF THE INVENTION

1. Field of the Invention

The present invention relates to apparatuses, methods, and systems for simulating low and/or high intensity exercise. More particularly, the present invention relates to an exercise mimetic device for simulating low and/or high intensity exercise using low intensity electrical stimulation to generate low intensity muscle contractions such as a wearable garment that preferably imitates exercise by eliciting low grade muscle contractions in several of the larger skeletal muscle groups in the body. The apparatus of various embodiments of the present invention is a neuromuscular electrical stimulation (NMES) device/garment with a control unit that is wirelessly connected to and controls a stimulator unit that generates and transmits a low intensity electrical stimulation within certain unique parameters. In various embodiments, the NMES device/garment is for treating conditions including but not limited to obesity, obesity related conditions such as diabetes, muscle toning, and/or other conditions benefitted by exercise. In various embodiments, the NMES device/garment is an over the counter (OTC) NMES device/garment.

2. General Background of the Invention

It is known in the art that only low intensity exercise burns fat and high intensity exercise burns glycogen and then muscle. Research on skeletal muscle mitochondrial metabolism of fats from Arend Bonen has revealed many important things regarding fat metabolism, among which are: 1) that skeletal muscle mitochondria could be stimulated to burn fat through electrical or neuromuscular stimulation (NMES), 2) that overweight/obese people had normal functioning mitochondria but only half as many as normal weight people, 3) that prolonged, low intensity exercise increased the number of mitochondria and increased their ability to use fat for energy. In applicants' treatment of over 170,000 overweight/obese patients over 34 years, the applicants are well aware that it was harder in the short term to get patients to exercise, that some extremely obese and physically impaired patients could not exercise, and that weight maintenance was a function primarily of exercise (as confirmed by most studies). Furthermore, in Applicants' view, the chief reason patients did not exercise was their busy schedule and resultant lack of time.

As far back as the 1950's, East German and Russian scientists pioneered the use of NMES for muscle mass and strength development with excellent results. Subsequent research showed that NMES was at least as effective as free weight training for mass and strength development (Type 2 muscle fiber) development, and was now used with paralyzed patients to inhibit muscle wasting and atrophy. Several studies were also done on the fat burning potential of NMES. A group in Ireland had done this by mimicking shivering through a very complex stimulation pattern and had marketed some products such as an ab belt, a butt belt and a thigh belt—with little market penetration. However, the definitive study on fat burning potential was done by Miao-Ju Hsu, et. al in a 2011 Sensors article which showed that low intensity exercise in 4 large muscle groups bilaterally for 30 minutes resulted in an (extrapolated) 75 kcal/hr increase in metabolism from baseline, much or most of it fat tissue by the drop in measured respiratory quotient. Realizing that more fat could be burned by 1) including more muscle groups and 2) adjusting the NMES parameters to better recruit and stimulate primarily Type 1 mm fibers without associated fatigue, and 3) increasing duration of stimulation, a significant fat burning effect could be achieved.

The health benefits of NMES/TENS go beyond just loss of excess fat tissue and the concomitant comorbid factors associated with overweight and obesity (Metabolic Syndrome, dyslipidemia, hypertension, increased risk of cardiovascular events, Nonalcoholic Fatty Liver Disease, Nonalcoholic Steatohepatitis, diabetes and glucose intolerance, insulin resistance, joint and bone degeneration, various forms of cancer, etc). The well documented metabolic improvements associated with prolonged low intensity exercise noted in the medical literature actually shows that for Type 2 diabetics, exercise is more effective than the diabetic drug metformin in controlling glucose.

In regard to NMES devices, there are existing devices on the market, all with shortcomings.

After using multiple NMES units, we found that our needs for a small, multi-channel, long running unit with unique parameters were not met by any of the currently available units on the market. From the units we have used, the large EV906 was completely programmable, too heavy and only supplied 4 channels supporting 8 electrodes. Very small units currently on the market are not optimal for use as an exercise mimetic device. Furthermore, these units only provide 4 channels and were not dependable.

One major problem with prior art NMES devices is that the screen and controls are part of the unit, which presents a serious problem when changing various parameters during the course of the stimulation period, as frequently occurs during the day in regards to changing the intensity of the current provided by the different channels on the NMES unit. To adjust the settings, one must be able to actually see the screen and the control buttons housed in the single NMES unit. This requires maneuvering the unit from wherever it is secured on the body (often under one's clothing) such that the unit's screen and buttons can be viewed and manipulated. This can be problematic. Not only does this repositioning present the user with having to access and then reposition the NMES unit from wherever it is secured on the person, it also requires the presence of “slack” in the wires to accomplish this movement, which often results in the wires being caught on objects and disconnected.

One example of a currently marketed wireless NMES unit on the market is the large COMPEX® wireless units, models SP 6.0 and SP 8.0. The COMPEX® unit is made of two separate and distinct entities: 1) a central controller that acts as both an electrical generator and controller, which wirelessly transmits the appropriate electrical parameters; and 2) a module, transmitting the signal from the controller to an electrode which is attached to each module. Two modules correspond to each channel in the controller, for a total of eight wireless modules. Even though this setup eliminates the wires from the controller to the electrodes, there are wires still connecting the two modules which connect to the electrodes. These receiver modules must also be charged, along with the controller. The modules are also very bulky, expensive and not practical for all-day wear under normal clothing.

The following references are incorporated herein by reference: U.S. Pat. Nos. 4,586,495; 5,476,441; 5,628,722; 5,724,996; 6,151,528; 6,341,237; 6,728,577; 6,760,629; 6,885,896; 7,069,089; 7,257,448; 7,747,327; 8,265,763; 8,494,658; D472,460; U.S. Publication Document Nos. 2002/0058972; 2002/0133195; 2004/0172097; 2005/0055067; 2006/0247733; 2008/0097530; 2010/0185259; 2010/0262052; 2010/0273614; 2010/0312306; 2011/0071595; 2011/0295339; 2012/0116477; 2012/0172940; 2012/0203156; PCT Publication No. WO 02/068040; WO 02/074109; WO 2006/038235; WO 2006/113802; WO 2006/121463; WO 2011/153213; WO 2014/089331; and Foreign Patent Publications EP021904 and DE202011109226. The following publications are hereby incorporated herein by reference: http://www.slendertone.com/en-us/about-the-slendertone.html; http://www.slendertone.com/en-us/toning-for-women/bottom-1.html; http://www.slendertone.com/en-us/toning-for-women/abs-belts.html; http://www.slendertone.com/en-us/toning-for-women/arms-1.html; http://www.slendertone.com/en-us/toning-for-women/bundles-1.html; http://www.compex.info/en_UK/Compex_categories_new.html; http://www.compex.info/en_UK/72532-SP-6-0.html; http://www.compex.info/en_UK/72532-SP-6-0.html; http://www.gizmag.com/antelope-electrode-suit/36532/?utm_source=Gizmag+Subscribers&utm_campaign=41988df3d6-UA-2235360-4&utm_medium=email&utm_term=0_65b67362bd-41988df3d6-91569849; http://wls.club/#testimonials; http://www.gizmag.com/antelope-electrode-suit/36532/pictures#4; http://www.blogsrelease.com/index.php?module=Board&id=5107; Banerjee et al., Prolonged electrical muscle stimulation exercise improves strength and aerobic capacity in healthy sedentary adults. J. Appl. Physiol. 2005, 99, 2307-2311; Banerjee et al., Electrical stimulation of unloaded muscles causes cardiovascular exercise by increasing oxygen demand. Eur. J. Cardiovasc. Prev. Rehabil. 2005, 12, 503-508; and Hsu et al., Effect of Neuromuscular Electrical Muscle Stimulation on Energy Expenditure in Healthy Adults. Sensors 2011, 11, 1932-1942.

### BRIEF SUMMARY OF THE INVENTION

The present invention relates to apparatuses, methods, and systems for simulating low and/or high intensity exercise. More particularly, the present invention relates to an exercise mimetic device for simulating low and/or high intensity exercise using low intensity electrical stimulation to generate low intensity muscle contractions such as a wearable garment that preferably imitates exercise by eliciting low grade muscle contractions in several of the larger skeletal muscle groups in the body. The apparatus of various embodiments of the present invention is a neuromuscular electrostimulation (NMES) device/garment with a control unit that is wirelessly connected to and controls a stimulator unit that generates and transmits a low intensity electrical stimulation within certain unique parameters. In various embodiments, the NMES device/garment is for treating conditions including but not limited to obesity, obesity related conditions such as diabetes, muscle toning, and/or other conditions benefitted by exercise. In various embodiments, the NMES device/garment is an OTC NMES device/garment.

The challenges applicant confronted and solved in the present invention for a garment worn anywhere from several hours up to 14 hours under normal clothing included: 1) securing electrodes to different body parts, 2) ease of donning the garment, 3) proper electrode choice, 4) ease of bathroom access, 5) whole suit vs. half suit, 6) material(s), 7) ease of cleaning, 8) what to do with all those loose wires (a critical and challenging problem with several solutions).

In various embodiments, the NMES device/garment has a crotch cut out or opening in the crotch area to allow for convenient access to the restroom without removing the garment.

In various embodiments, the NMES device/garment has separate upper and lower pieces.

In various embodiments, the NMES device/garment has zippers on outer aspects of both legs.

In various embodiments, the NMES device/garment has stretchable material channels sewn for incorporating wires.

In various embodiments, the NMES device/garment comprises means for attaching with electrodes for muscular stimulation, where the means allow for the electrodes to be affixed to the NMES device/garment at various positions.

In various embodiments, the NMES device/garment is a device/garment including combinations of embodiments of the present invention.

In various embodiments, electrodes are removably attached to the garment.

In various embodiments, the wiring is removably attached to the garment.

In various embodiments, the wiring is placed within compartments such as within the bore/channel of tubing such that the compartments are removably attached to the garment.

In various embodiments, the NMES unit(s) is/are removably attached to the garment.

In various embodiments, the electrodes, wiring, and the NMES unit(s) are detached from the garment in order to allow for cleaning of the garment.

In various embodiments, the electrodes, wiring, and the NMES unit(s) are reattached to the garment after cleaning.

In various embodiments, the garment includes separate upper and lower sections.

In various embodiments, the upper and lower section can be secured together when being worn by a user.

In various embodiments, the garment covers both the upper and lower sections of a user.

In various embodiments, the electrodes wiring, and NMES unit(s) form a NMES system that is removably attached to the garment in a manner to stimulate muscle contractions when engaged.

In various embodiments, the NMES system is removably attached to the inner surface of the garment in a manner to stimulate muscle contractions when engaged.

In various embodiments, the NMES system is removably attached to the inner surface of the garment in a manner to stimulate muscle contractions when engaged. In various embodiments, the garment includes a plurality of opening to allow the electrodes of the NMES system to contact the skin/surface of a wearer in a manner to stimulate muscle contractions when engaged.

In various embodiments, the wires, electrodes, and NMES unit(s) of the NMES system can be removably attached to either the inner or outer surface of the garment.

While certain novel features of this invention shown and described below are pointed out in the annexed claims, the invention is not intended to be limited to the details specified, since a person of ordinary skill in the relevant art will understand that various omissions, modifications, substitutions and changes in the forms and details of the device illustrated and in its operation may be made without departing in any way from the spirit of the present invention. No feature of the invention is critical or essential unless it is expressly stated as being “critical” or “essential.”

### BRIEF DESCRIPTION OF THE SEVERAL VIEWS OF THE DRAWINGS

For a further understanding of the nature, objects, and advantages of the present invention, reference should be had to the following detailed description, read in conjunction with the following drawings, wherein like reference numerals denote like elements and wherein:

FIG. 1 is a front view of the outer surface of an apparatus of an embodiment of the present invention showing a garment with upper and lower parts (or upper and lower body sections), a crotch cut out, and zippers;

FIG. 2 shows a back view of the outer surface of an apparatus of an embodiment of the present invention showing a garment with upper and lower parts (or upper and lower body sections), a crotch cut out, and zippers;

FIG. 3 is a front view of the inner surface of an apparatus of an embodiment of the present invention showing the placement of the electrodes on the surface of a user's body, wiring, and NMES unit(s);

FIG. 4 is a back view of the inner surface of an apparatus of an embodiment of the present invention showing the placement of the electrodes on the surface of a user's body and wiring;

FIG. 5 is a front close-up view of the inner surface of the upper part (or upper body section) of an apparatus of an embodiment of the present invention showing the placement of the electrodes on the surface of a user's body, wiring, and NMES unit(s);

FIG. 6 is a back close-up view of the inner surface of the upper part (or upper body section) of an apparatus of an embodiment of the present invention showing the placement of the electrodes on the surface of a user's body and wiring;

FIG. 7 is a front close-up view of the inner surface of the lower part (or lower body section) of an apparatus of an embodiment of the present invention showing the placement of the electrodes on the surface of a user's body, wiring, and NMES unit(s);

FIG. 8 is a back close-up view of the inner surface of the lower part (or lower body section) of an apparatus of an embodiment of the present invention showing the placement of the electrodes on the surface of a user's body and wiring;

FIG. 9 is a reverse close-up view of the inner surface of the upper part (or upper body section) of an apparatus of an embodiment of the present invention showing that the electrodes, wiring, and NMES unit are removably attached to the garment.

FIG. 10 shows a view of a screen of a controller unit of an apparatus of an embodiment of the present invention;

FIGS. 11-28 are views of an apparatus of an embodiment of the present invention showing a general user interface concept;

FIG. 29 is a front view of the inner surface of an apparatus of an embodiment of the present invention showing the placement of the electrodes on the surface of a user's body, wiring, and NMES unit(s);

FIG. 30 is a front view of an apparatus of an embodiment of the present invention showing a garment for the lower body with and an example of a lower NMES body garment with the placement of the electrodes on the surface of a user's body, wiring, and NMES unit(s);

FIG. 31 is a photo showing a front view of a controller unit and stimulator/electrical generating unit of an apparatus of an embodiment of the present invention;

FIG. 32 shows a view of a screen of a controller unit and stimulator/electrical generating unit of an apparatus of an embodiment of the present invention;

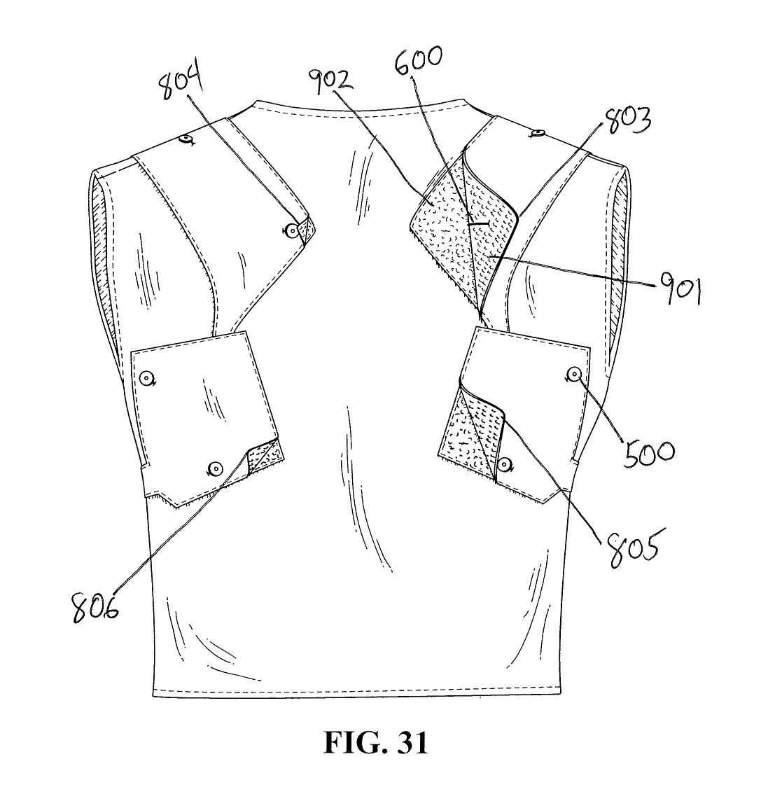

FIG. 31 is a back close-up view of the outer surface of the upper part (or upper body section) of an apparatus of an embodiment of the present invention showing the placement of the covers for the electrodes on the surface of a user's body and wiring;

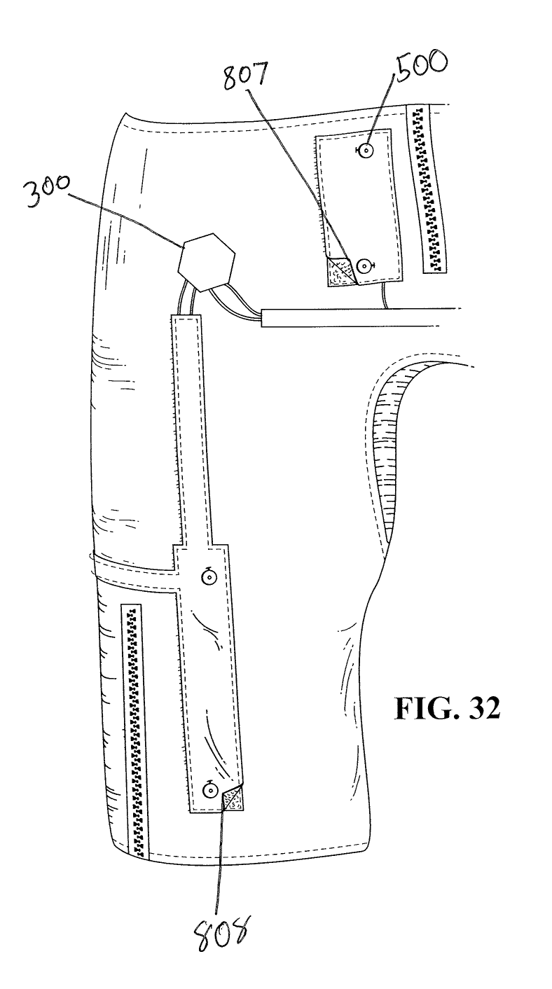

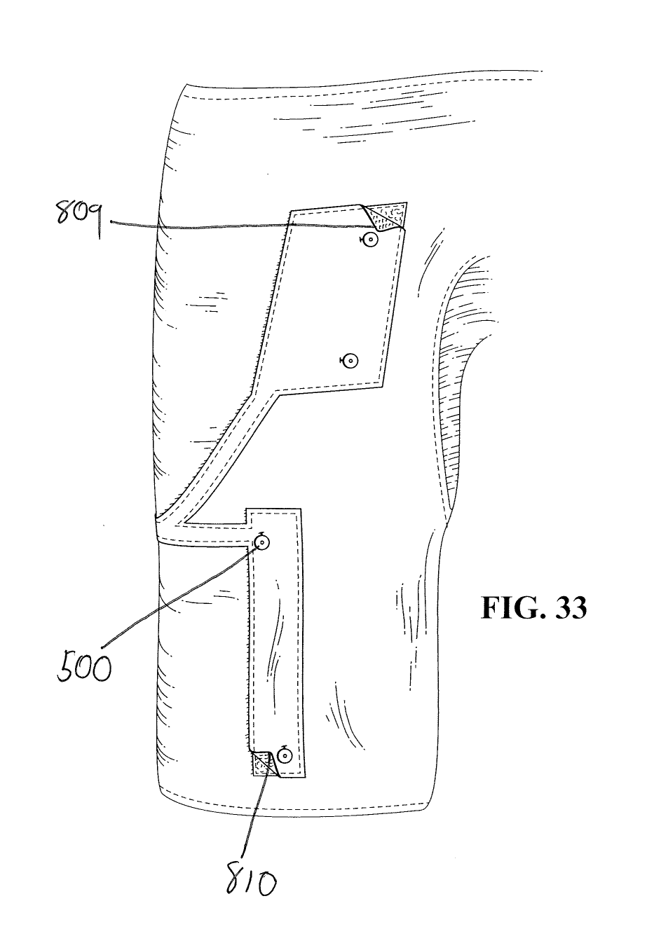

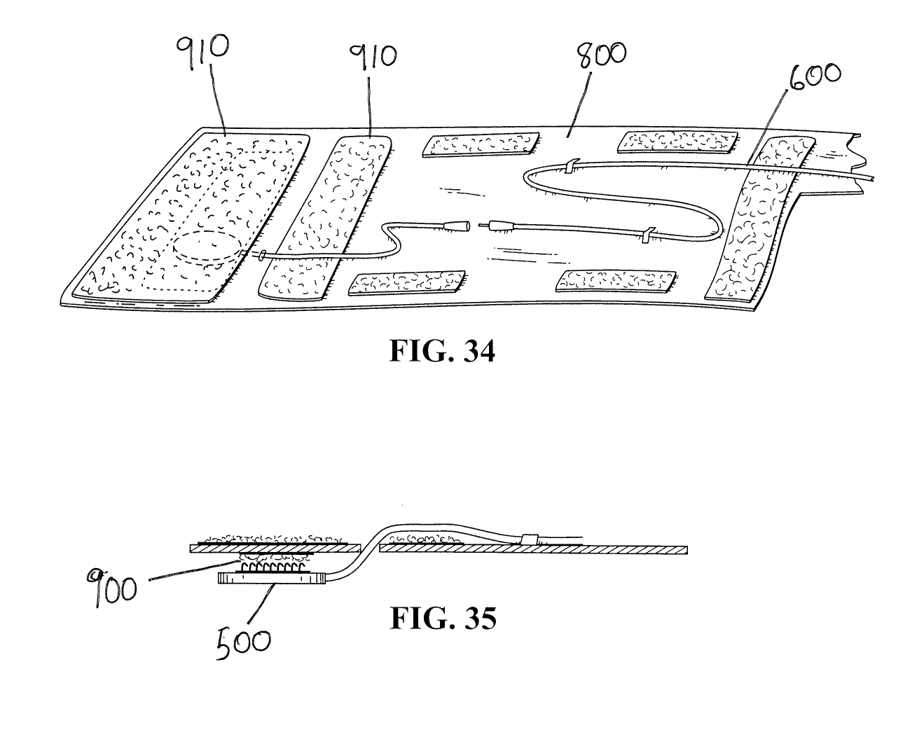

FIG. 32 is a front close-up view of the outer surface of the lower part (or lower body section) of an apparatus of an embodiment of the present invention showing the placement of the covers for the electrodes on the surface of a user's body and wiring;

FIG. 33 is a back close-up view of the outer surface of the lower part (or lower body section) of an apparatus of an embodiment of the present invention showing the placement of the covers for the electrodes on the surface of a user's body and wiring;

FIG. 34 is a top view of a compartment of an apparatus of the present invention showing electrodes and wires removably attached to the sheet; and

FIG. 35 is a top view of a compartment of an apparatus of the present invention showing electrodes and wires removably attached to the sheet.

### DETAILED DESCRIPTION OF THE INVENTION

The present invention relates to apparatuses, methods, and systems for simulating low intensity exercise. More particularly, the present invention relates to an exercise mimetic device for simulating low intensity exercise using low intensity electrical stimulation to generate low intensity muscle contractions such as a wearable garment that preferably imitates exercise by eliciting low grade muscle contractions in several of the larger skeletal muscle groups in the body. The apparatus of various embodiments of the present invention is a neuromuscular electrostimulation (NMES) device/garment with a control unit that is wirelessly connected to and controls a stimulator unit that generates and transmits a low intensity electrical stimulation within certain unique parameters. In various embodiments, the NMES device/garment is for treating conditions including but not limited to obesity, obesity related conditions such as diabetes, muscle toning, and/or other conditions benefitted by exercise. In various embodiments, the NMES device/garment is an OTC NMES device/garment. The challenges applicant confronted and solved in the present invention for a garment worn anywhere from several hours up to 14 hours under normal clothing included: 1) securing electrodes to different body parts, 2) ease of donning the garment, 3) proper electrode choice, 4) ease of bathroom access, 5) whole suit vs. half suit, 6) material(s), 7) ease of cleaning, 8) what to do with all those loose wires (a critical and challenging problem with several solutions).

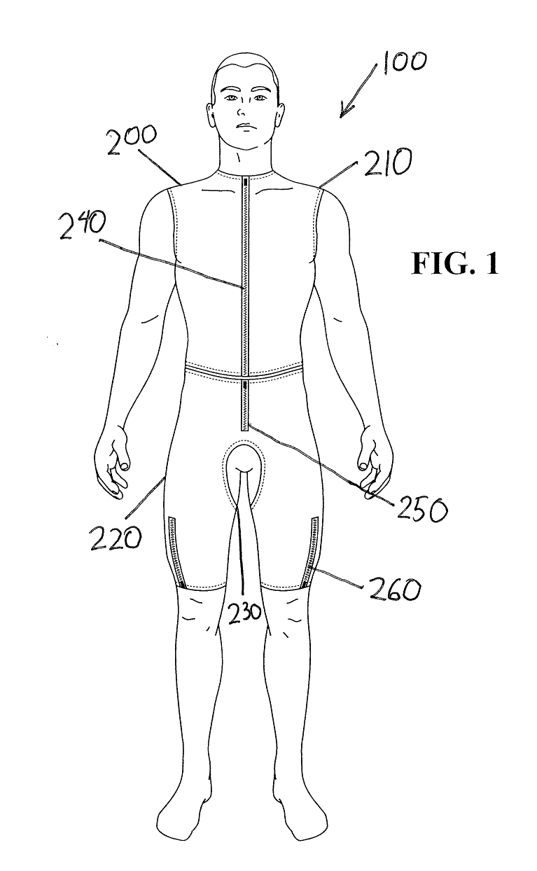

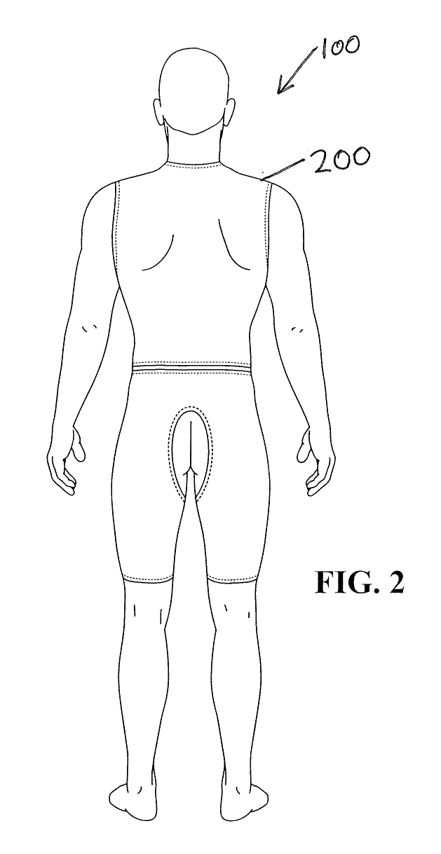

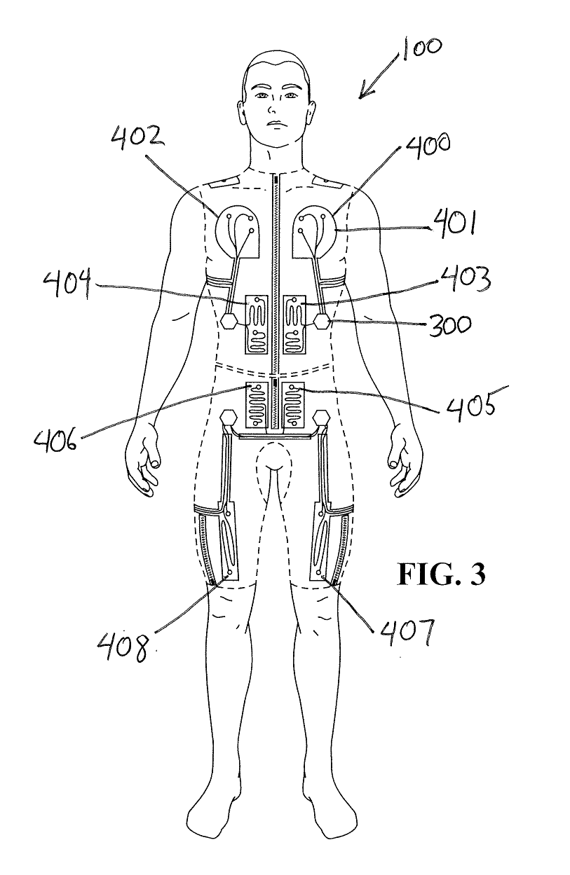

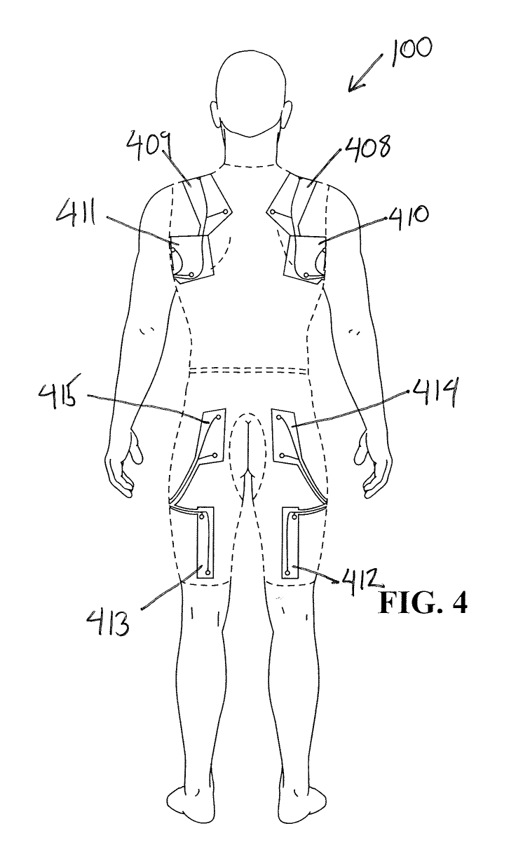

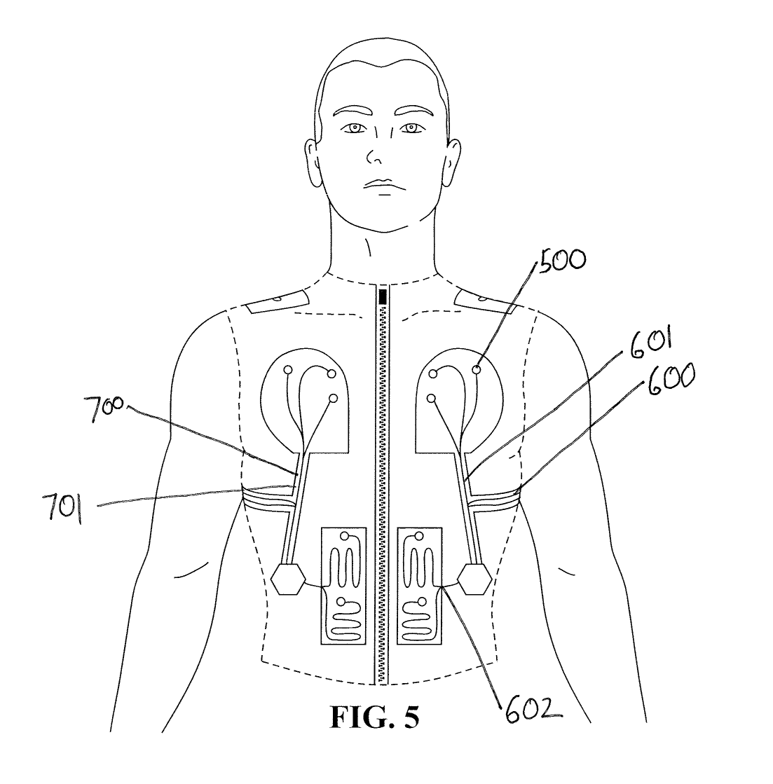

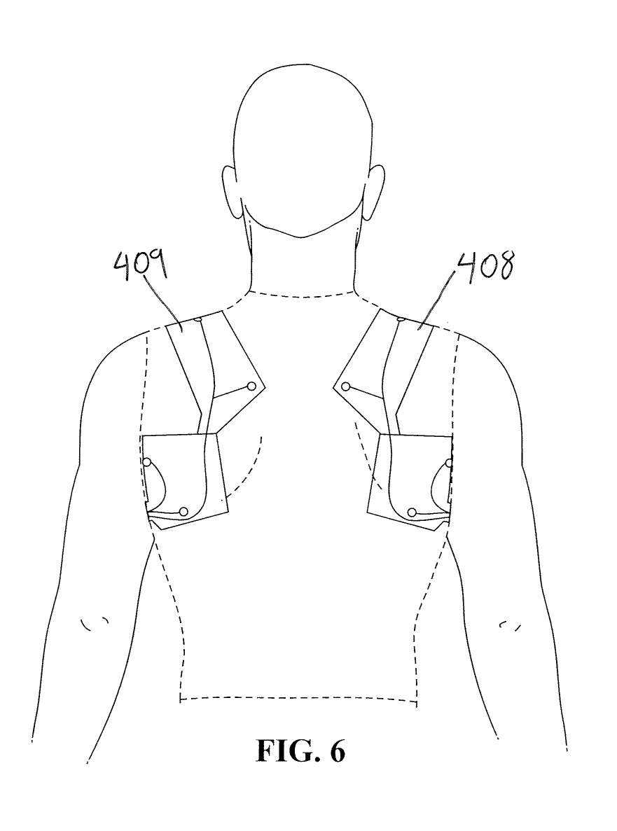

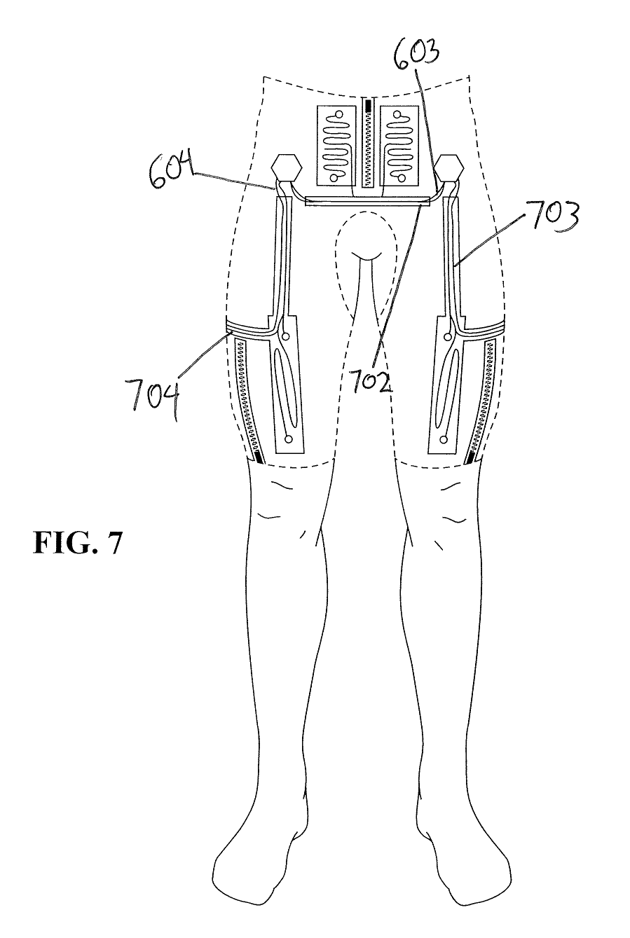

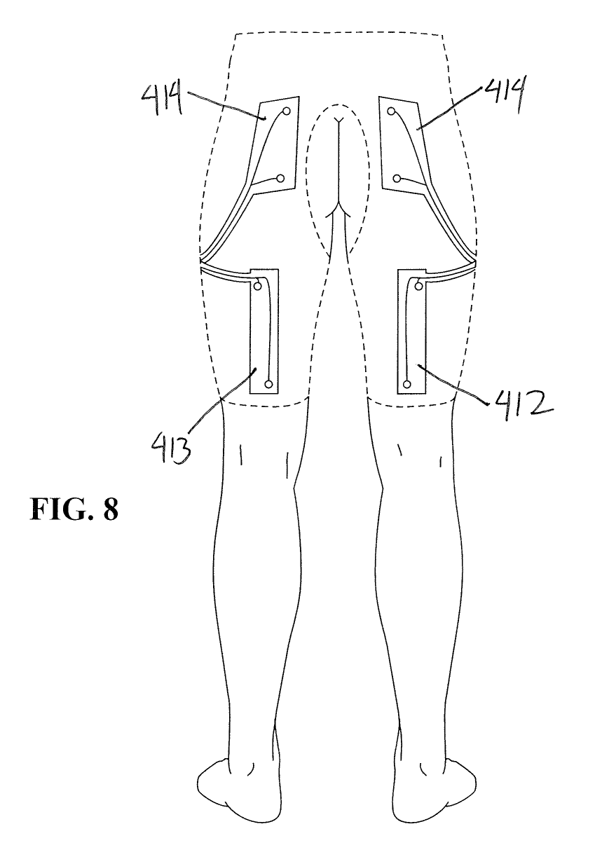

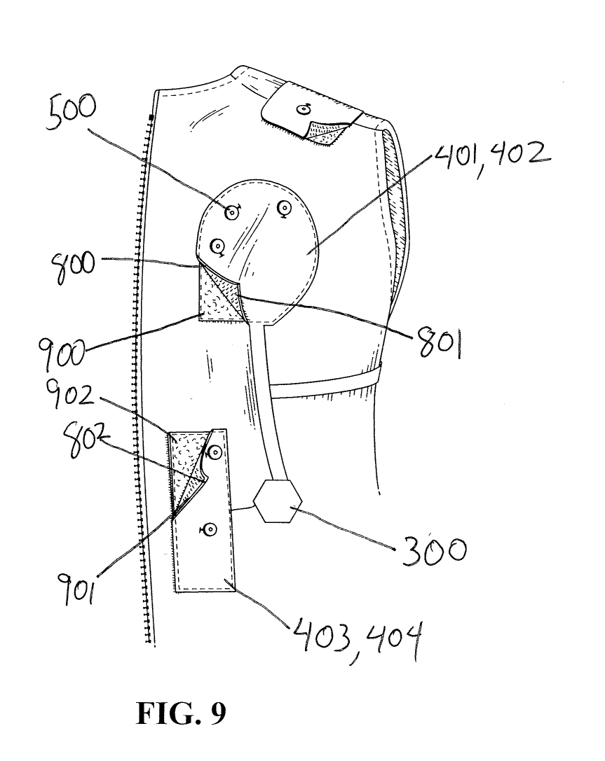

In various embodiments of the present invention, the exercise mimetic device 100 is a NMES exercise suit. FIGS. 1-9 and 31-35 show various views of an apparatus 100 of various embodiments of the present invention. The NMES exercise suit preferably generates low intensity electrical stimulation within preferable and unique parameters to preferably generate low intensity muscle contractions that preferably simulate low intensity exercise. By eliciting low intensity muscle contractions that mimics low intensity exercise, the NMES device 100 is preferably an exercise mimetic device. In various embodiments, the low grade muscle contractions are preferably accomplished through stimulation of skeletal muscle mitochondria.

In various embodiments of the present invention, the apparatus 100 preferably includes two components: (1) one or several garments 200 preferably having a unique design; and (2) a custom made, one-of-a-kind NMES unit and several necessary support components.

In various embodiments, the NMES device/garment 100 is a full body suit. In another embodiment, the NMES device/garment is a lower body suit. In another embodiment, the NMES device/garment is a “high waist” type of garment 210, preferably extending from just under the ribs down.

In various embodiments, the NMES device/garment is preferably a modified wetsuit-like garment such as a one piece suit. The one piece suit preferably covers a patient's upper and lower body and is preferably similar to currently available OTC wetsuits. The one piece suit preferably includes different patterns. In various embodiments, the wetsuit pattern is preferably purchased and/or downloaded online.

In various embodiments of the present invention, the NMES device/garment has a crotch cut out 230 that preferably allows for easy perineal/anal access for bathroom usage while continuously worn without the need to remove said garment/device or opening in the crotch area 230 to allow for convenient access to the restroom without removing the garment. The NMES device/garment of various embodiments of the present invention preferably allows a wearer to: (1) preferably use the bathroom while wearing the NMES device/garment; and/or (2) preferably wear regular underwear concurrently with the NMES device/garment.

In various embodiments of the present invention, the NMES device/garment is preferably a variation to the wet suit embodiment, wherein the NMES device/garment preferably has straps to preferably secure an upper part of the NMES device/garment onto the trapezius of a wearer, preferably similar to that of a tank top or undershirt. This embodiment preferably makes the NMES device/garment cooler temperature wise.

In various embodiments of the present invention, the NMES device/garment is made up of neoprene. Alternatively, the NMES device/garment may be made of but not limited to: spandex, spandex containing material, 2-way or 4-way stretchable material or other stretchable material. More preferably, the neoprene can be of a thin density and/or has a light weight. Most preferably, the neoprene can be of the thinnest density and/or has a lightest weight possible. The neoprene of this embodiment preferably: does not tear; does not easily lose its elasticity; does not easily lose its shape and/or integrity; and/or is easy to clean. The neoprene can be preferably antimicrobial and/or comprises means for eliminating odors. In various embodiments, the NMES device/garment is made up of a neoprene with various sizes and patterns of holes punched through (Airprene).

In various embodiments, the NMES device/garment has separate upper 210 and lower 220 pieces that preferably accommodates various body sizes and shapes. More preferably, The NMES device/garment of this embodiment accommodates various sizes and shapes of buttocks and/or breasts and/or leg circumference and/or abdominal circumference.

In various embodiments, the NMES device/garment has zippers on outer aspects of both legs and in the front of the main body. In this embodiment, the zippers are preferably located on outer aspect of both legs. In this embodiment, the zippers are preferably located on the front of the NMES device/garment. The zippers preferably aid a wearer in donning the garment such as larger patients and patients with very large legs. In various embodiments, the wearers may have a condition that is medically termed lipedema.

In various embodiments, electrodes 500 are removably attached to the garment 200.

In various embodiments, the wiring is removably attached to the garment 200.

In various embodiments, the wiring is placed within compartments 400 such as within the bore/channel 700 of tubing such that the compartments 400 are removably attached to the garment 200.

In various embodiments, the NMES unit(s) is/are removably attached to the garment 200.

In various embodiments, the garment 200 has channels 700 through which the wires 600 can be removable placed within.

In various embodiments, the electrodes 500, wiring, and the NMES unit(s) are detached from the garment 200 in order to allow for cleaning of the garment 200.

In various embodiments, the electrodes 500, wiring, and the NMES unit(s) are reattached to the garment 200 after cleaning.

In various embodiments, the garment 200 includes separate upper and lower sections.

In various embodiments, the upper 210 and lower 220 section can be secured together when being worn by a user.

In various embodiments, the garment 200 covers both the upper and lower sections of a user.

In various embodiments, the electrodes 500, wiring, and NMES unit(s) for a NMES system that is removably attached to the garment 200 in a manner to stimulate muscle contractions when engaged.

In various embodiments, the NMES system includes an upper NMES system removably attached to the upper section of the garment and a lower NMES system removably attached to the lower section of the garment 200.

In various embodiments, the NMES system is removably attached to the inner surface of the garment 200 in a manner to stimulate muscle contractions when engaged.

In various embodiments, the NMES system is removably attached to the inner surface of the garment 200 in a manner to stimulate muscle contractions when engaged. In various embodiments, the garment 200 includes a plurality of opening to allow the electrodes 500 of the NMES system to contact the skin/surface of a wearer in a manner to stimulate muscle contractions when engaged.

In various embodiments, the wires 600, electrodes 500, and NMES unit(s) of the NMES system can be removably attached to either the inner or outer surface of the garment 200.

Compartment 400 includes a plurality of compartments 401, 402, 403, 404, 405, 406, 407, 408, 409, 410, 412, 413, 414, 415.

Wires 600 includes a plurality of wires 601, 602, 603, 604.

Channel 700 includes a plurality of channels 701, 702, 703, 704, 705.

Sheets 800 includes a plurality of sheets 801, 802, 803, 804, 805, 806, 807, 808, 809, 810.

In various embodiments, the apparatus 100 comprises a means for attaching electrodes 500, wiring, and NMES unit(s) to the garment 200 for muscular stimulation. In various embodiments, the means allow for the electrodes 500, wiring, and NMES unit(s) to be affixed to the garment 200 at various positions.

In various embodiments, the electrodes 500, wiring, and NMES unit(s) are contained within compartments 400 such as bags having an outer surface that can be removably attached to the inner surface of the garment 200.

In various embodiments, the compartments 400 containing the electrodes 500 have opening allowing for the electrodes 500 to be positioned on the skin of a user/wearer.

In various embodiments, the wiring is placed within compartments 400 such as within the bore/channel 700 of tubing such that the compartments 400 are removably attached to the garment 200.

In various embodiments, the compartment 400 includes a plurality of compartments for removably attaching the wires 600, electrodes 500, and NMES unit(s) of the NMES system.

In various embodiments, the garment 200s have channels 700 through which the wires 600 can be removable placed within.

In various embodiments, the electrodes 500, wiring, and NMES unit(s) directly and removably attach to the garment 200. The electrodes 500, wiring, and NMES unit(s) of various embodiments may have a surface with a means for directly attaching the garment 200.

In various embodiments, the attachment means is a hook and loop fastener 900 (i.e., VELCRO®) with the loops preferably on the garment 200 and hooks on the electrodes 500, wiring, and NMES unit(s).

In various embodiments, the attachment means is a hook and loop fastener 900 (i.e., VELCRO®) with the loops preferably on the electrodes, wiring, and NMES unit(s) and hooks on the garment 200.

In various embodiments, the compartment 400 can be formed by a sheet 800 that is attachable to the garment 200 to form the compartment 400 and having an outer surface facing the skin of a wearer and an inner surface facing the garment 200.

The sheet 800 of various embodiments is thin, lightweight, and collapsible and can be made up of a flexible material. In various embodiments, the flexible material including, for example, laminated cotton, vinyl fabrics, cotton, polyester, nylon, rayon, wool, gabardine, vinyl, microfibers and high tech fabrics. In other embodiments, the flexible material can be the same plastic type fabric used on raincoats that can be fold up into an thimble.

In various embodiments, the sheet 800 is attachable to the garment 200. The attachment can be accomplished, for example, with an attachment means including a hook and loop fastener 900 (i.e. VELCRO®), silicone, or an elastic or sticky substance. In various embodiments, the attachment means can be attached to the sheet 800 by means such as, for example, glue or heat stamping.

In various embodiments, a second attachments means can be used to trap excess length of wires 600 to the inner surface of the sheet 800. The attachment can be accomplished, for example, with an attachment means including a hook and loop fastener 900 (i.e. VELCRO®), silicone, or an elastic or sticky substance. In various embodiments, the attachment means can be attached to the sheet 800 by means such as, for example, glue or heat stamping.

In various embodiments, the wires 600 of any embodiment of the NMES System has a length to avoid excess lengths and/or to allow for slack to allow them to be moved easier.

The wires 600 of any embodiment of the NMES System may also include connectors to connect with the electrodes 500 and NMES unit. The connectors can include, for example, Molex connectors.

In various embodiments, the sheet 800 can include a hole such that electrodes 500 removably attached to the outer surface can connect to the wires 600 removably attached to the inner surface of the sheet 800. The electrode(s) of various embodiments can include wiring with a connector such that the wiring and connector can be threaded through the hole to connect to the wire. In various embodiments, the hole at the left side of the sheet 800 can allow the wire of the electrode to pass through from the “GARMENT-SHEET 800” (outer surface) area to the “SHEET-SKIN” (inner surface) area.

In various embodiments as shown in Figures, there is at each end a Velcro loop strip under the sheet (skin side) to which the electrode (with its Velcro hooks on the back [non-skin side]) (inner surface) sticks to. In other embodiments, the loop strips can be moved in any manner include, for example, higher, lower, left or right, to accommodate different heights of folks (probably need less than 2 inches of movement). In other embodiments, the right side is the same “sheet-skin” side strip of velcro loops affixed to the sheet 800. The right side is the “channel” can lead wires 600 to the NMES.

In various embodiments, the channel 700 can be made up of the same materials as the sheet 800.

In various embodiments, the NMES device/garment can have stretchable material channels sewn for incorporating wires 600. The channels are preferably sewn into/onto parts of the suit to preferably and completely cover all wires 600 as they extend on the outer, non-skin surface of the garment 200 on their way to connect to a NMES stimulator unit. This embodiment preferably prevents the wires 600 from snagging on something.

In various embodiments, the electrodes 500 are positioned in a manner to stimulate any muscle group to contract.

In various embodiments, the electrodes 500 are positioned in a manner to stimulate a major muscle group to contract. In other embodiments, the electrodes 500 are positioned in a manner to stimulate a plurality of major muscle groups to contract.

In various embodiments, the electrodes 500 are positioned in a manner to stimulate a minor muscle group to contract. In other embodiments, the electrodes 500 are positioned in a manner to stimulate a plurality of minor muscle groups to contract.

In various embodiments, the electrodes 500 are positioned in a manner to stimulate a major and a minor muscle group to contract.

In various embodiments, electrodes 500 are positioned in a manner to stimulate the abdominal muscles including the upper and lower abdominal muscles to contract. The electrodes 500 of various embodiments can straddle the edge of the abdominal muscles and can be in a compartment/bag 400 such as abdominal bag.

In various embodiments, electrodes 500 are positioned in a manner to stimulate the gastrocnemius muscle to contract. The electrodes 500 of various embodiments can straddle the edge of the gastrocnemius muscle and can be in a compartment/bag 400 such as gastrocnemius bag.

In various embodiments, electrodes 500 are positioned in a manner to stimulate the bilateral pectoralis major muscles to contract. The electrodes 500 of various embodiments can straddle the edge of the abdominal and can be in a compartment/bag 400 such as pectoralis bag.

In various embodiments, electrodes 500 are positioned in a manner to stimulate the Latissimus dorsi muscles to contract. The electrodes 500 of various embodiments can straddle the edge of the Latissimus dorsi muscles and can be in a compartment/bag 400 such as a Latissimus dorsi bag.

In various embodiments, electrodes 500 are positioned in a manner to stimulate the trapezius muscles to contract. The electrodes 500 of various embodiments can straddle the edge of the trapezius muscles and can be in a compartment/bag 400 such as a trapezius bag.

In various embodiments, wires 600 connecting the NMES Unit to the electrodes 500 are contained within channels 700. The channels 700 of various embodiments can contain a plurality of wires 600. For example in various embodiments, the channel 700 contains two wires 600 with one wire connecting the NMES unit to Latissimus dorsi muscles and another wire connected the NMES unit to the trapezius muscles. In another example of various embodiments, the channel 700 shown contains two wires 600 with one wire connecting the NMES unit to Latissimus dorsi muscles and another wire connected the NMES unit to the trapezius muscles (same bilaterally).

In various embodiments, wires 600 connecting the NMES unit to the electrodes 500 for stimulating the trapezius muscles does not cross the wires 600 for stimulating the Latissimus dorsi muscles. These wires 600 can extend side-by-side in a channel 700 running from the front of a wearer to the back of the wearer.

In various embodiments, electrodes 500 are positioned in a manner to stimulate the Quadriceps femoris muscles to contract. The electrodes 500 of various embodiments can straddle the edge of the Quadriceps femoris muscles and can be in a compartment/bag 400 such as a Quadriceps bag.

In various embodiments, electrodes 500 are positioned in a manner to stimulate the Hamstring muscles to contract. The electrodes 500 of various embodiments can straddle the edge of the hamstring muscles and can be in a compartment/bag 400 such as a hamstring bag.

In various embodiments, electrodes 500 are positioned in a manner to stimulate the gluteus muscles to contract. The electrodes 500 of various embodiments can straddle the edge of the gluteus muscles and can be in a compartment/bag 400 such as a gluteus bag.

In various embodiments, the connection between the stimulator and electrodes 500 may include a conductive material which is part of the garment or affixed to the garment (sewn, glued, heat stamped, etc.)

FIGS. 34 and 35 highlight various embodiments of the present invention.

In various embodiments as shown in FIGS. 34 and 35, wire for electrode to pass through to “skin” side of sheet/bag 400.

In various embodiments as shown in FIGS. 34 and 35, there is a hole to pass electrode wire through.

In various embodiments as shown in FIGS. 34 and 35, a hook strip to attach to corresponding loop strip on garment.

In various embodiments as shown in FIGS. 34 and 35, there is a Male plug on electrode.

In various embodiments as shown in FIGS. 34 and 35, there is a Female plug on wire going to NMES.

In various embodiments as shown in FIGS. 34 and 35, there is a hook facing and attaching to the garment that “traps” and holds a wire.

In various embodiments as shown in FIGS. 34 and 35, there is Loop part of type strip on underside of plastic sheet/bag 400.

In various embodiments as shown in FIGS. 34 and 35, there is an Electrode with hooks on back.

In various embodiments as shown in FIGS. 34 and 35, there is a wire from electrode.

In various embodiments as shown in FIGS. 34 and 35, a silicon strip or “sticky” material similar to iPhone earbud wires or elastic strip to hold wire to the plastic sheet/bag 400. In other embodiment, the apparatus can have several.

In various embodiments as shown in FIGS. 34 and 35, there is a slightly stretchable, very thin material, as either 1) a single sheet, or 2) a 2 layer bag 400. In various embodiments, the sheet has a fastener type strips (either hook or loop) 901, 902 around border to hold the inner surface of the garment.

The apparatus 100 of various embodiments of the present invention is a NMES device/garment with a control unit that is wirelessly connected to and controls a stimulator unit that generates and transmits a low intensity electrical stimulation within certain unique parameters. The control unit preferably comprises a screen and controls that are preferably separate from the stimulator unit. The NMES device/garment preferably supports at least fifteen hours of continuous stimulation preferably with unique patterns. The NMES device/garment can preferably stimulate 10 to 16 different muscle groups.

In various embodiments of the present invention, the apparatus 100 is a NMES device/garment comprising a stimulator/electrical generating unit and a control unit.

FIG. 31 shows an example of a control unit of various embodiments of the present invention.

The stimulator/electrical generating unit is preferably a small, thin unit and preferably includes but is not limited to: a power source; a central processing unit (CPU); a means for recharging the unit; and a means for generating and transmitting electrical stimulation to electrodes 500. Alternatively, the stimulator/electrical generating unit may further include a separate means for wireless connection to a control unit.

The power source is preferably a rechargeable battery such as a cell phone or lithium battery.

The CPU is preferably a simple programmable CPU chip. In various embodiments, the chip includes wireless connection means such as BLUETOOTH® or Wi-Fi. Most preferably, the chip has 10 to 16 channels and preferably supports 20 to 32 electrodes. In various embodiments, the CPU chip is a Nordic BLE+MO processor.

The means for recharging the unit is preferably a micro or mini universal serial bus (USB) hub. The USB hub can be covered with an open and closeable rubber seal to enhance protection from water and or body fluids. The stimulator unit can be preferably housed in a small mesh pouch made for this on the right upper abdomen.

The separate means for wireless connection to a control unit can be preferably a BLUETOOTH® chip. In various embodiments, the chip includes wireless connection means such as BLUETOOTH® or Wi-Fi. Most preferably, the chip has 10 to 16 channels and preferably supports 20 to 32 electrodes.

The stimulator/electrical generating unit preferably has a length ranging from 1.5 to 4 inches (3.81 cm to 10.2 cm). More preferably, the stimulator/electrical generating unit has a length ranging from 1.5 to 3 inches (3.81 cm to 7.61 cm). Most preferably, the stimulator/electrical generating unit has a length ranging from 1.5 to 2.5 inches (3.81 cm to 6.35 cm). In various embodiments, the stimulator/electrical generating unit has a length that is preferably half the length of an IPOD NANO®.

The stimulator/electrical generating unit preferably has a width ranging from ⅛ to 1 inch (0.32 cm to 2.54 cm). More preferably, the stimulator/electrical generating unit has a width ranging from ⅛ to ¾ inch (0.32 cm to 1.9 cm). Most preferably, the stimulator/electrical generating unit has a width ranging from ⅛ to ½ inch (0.32 cm to 1.27 cm).

The stimulator/electrical generating unit preferably has a height ranging from ½ to 4 inches (1.27 cm to 10.16 cm). More preferably, the stimulator/electrical generating unit has a height ranging from ½ to 3 inches (1.27 cm to 7.61 cm). Most preferably, the stimulator/electrical generating unit has a height ranging from ½ to 2 inches (1.27 cm to 5.08 cm).

The frequency of the electrical stimulation pulse generated and transmitted by the stimulator unit for low intensity muscle contractions preferably ranges from 1 Hz to 40 Hz. More preferably, the frequency of the electrical stimulation pulse generated and transmitted by the stimulator unit for low intensity muscle contractions ranges from 3 Hz to 33 Hz. Most preferably, the frequency of the electrical stimulation pulse generated and transmitted by the stimulator unit for low intensity muscle contractions ranges from 4 Hz to 20 Hz.

The amplitude of the electrical current parameter of the electrical stimulation pulse generated and transmitted by the stimulator unit for low intensity muscle contractions preferably ranges from 5 mA to 120 mA. More preferably, the amplitude of the electrical current parameter of the electrical stimulation pulse generated and transmitted by the stimulator unit for low intensity muscle contractions ranges from 10 mA to 100 mA. Most preferably, the amplitude of the electrical current parameter of the electrical stimulation pulse generated and transmitted by the stimulator unit for low intensity muscle contractions ranges from 10 mA to 80 mA.

In various embodiments, the electrical current parameter of the electrical stimulation pulse generated and transmitted by the stimulator unit for low intensity muscle contractions is preferably of a sufficient amperage to preferably induce low intensity muscle contractions in an obese individual. More preferably, the electrical current parameter is able overcome and penetrate the insulative properties of subcutaneous fat to stimulate low intensity muscle contractions and not exceed the maximum amperage settings understood by the inventors to be common to all NMES units on the market. The present inventors understand that the maximum amperage settings common to all NMES units on the market is 120 mA.

The control unit preferably includes a user interface preferably having a touch screen, controls such as buttons to activate the unit, means for saving and selecting programs related to the parameters of the stimulation unit including but not limited to increasing/decreasing electrical intensity.

In various embodiments of the present invention, the control unit is a wearer's mobile communication device such as a cell phone or tablet-type device preferably having wireless connection means such as BLUETOOTH® and programming for connecting and controlling the stimulator/electrical generating unit such as an appropriate control app. This embodiment preferably avoids the cost of a separate control unit (development, production and sales) and preferably avoids carrying around a unit that may be lost. FIG. 1 shows a controller unit of an embodiment of the present invention. In various embodiments, the use of a communication device preferably allows high intensity stimulation with various frequencies, wavelengths and/or stimulation time parameters for preferably increasing muscle strength, size, or both. In various embodiments, high intensity stimulation can preferably be used by professional athletes, power lifters, and/or body builders.

The frequency of the electrical stimulation pulse generated and transmitted by the stimulator unit for high intensity muscle contractions preferably ranges from 40 Hz to 90 Hz. More preferably, the frequency of the electrical stimulation pulse generated and transmitted by the stimulator unit for high intensity muscle contractions ranges from 45 Hz to 80 Hz. Most preferably, the frequency of the electrical stimulation pulse generated and transmitted by the stimulator unit for high intensity muscle contractions ranges from 50 Hz to 76 Hz.

The amplitude of the electrical current parameter of the electrical stimulation pulse generated and transmitted by the stimulator unit for high intensity muscle contractions preferably ranges from 2 mA to 450 mA. More preferably, the amplitude of the electrical current parameter of the electrical stimulation pulse generated and transmitted by the stimulator unit for high intensity muscle contractions ranges from 5 mA to 400 mA. Most preferably, the amplitude of the electrical current parameter of the electrical stimulation pulse generated and transmitted by the stimulator unit for high intensity muscle contractions ranges from 10 mA to 350 mA.

In various embodiments, the NMES device/garment can be a single entity.

In various embodiments, the NMES device/garment includes a stimulator/electrical generating unit that is an NMES generator and a separate controller.

In various embodiments, the stimulator/electrical generating unit transmits its electrical signal via hard wires.

In various embodiments, the NMES device/garment is significantly more dependable and safer than prior art devices.

In various embodiments, the stimulator/electrical generating unit 10-16 channels supporting 20-32 electrodes.

The following are examples of needs addressed by an apparatus 100 of various embodiments of the present invention:

(1) a neuromuscular electrical stimulator (NMES);
(2) a separate control unit controlling various parameter of the NMES, connected to the NMES stimulator unit via a single wired connection and used to control and recharge battery of the NMES stimulator unit;
(3) Food and Drug Administration (FDA) approval; and
(4) an iOS and Android app to control the NMES stimulator via compatible devices in lieu of the controller.

The following is an example of the dimensions/characteristics of a stimulator/electrical generating unit of a NMES device/garment of various embodiments of the present invention:

(1) SIZE: Preferably as small as possible such as an IPOD NANO® size.
(2) POWER: Rechargeable battery
a. Rechargeable preferably via USB or similar to a USB or via wire from controller
b. Preferably providing a minimum of 16 hours of continuous service
   (3) CHANNELS: Preferably 16 channels supporting 2 electrodes per channel, each channel preferably corresponding to the two electrodes connected to a certain body part (i.e., Channel #1=corresponds to the positive and negative electrodes of the right hamstring group).
   (4) PROGRAMS: Preferably 5-6 stimulation programs preferably with the following constant/fixed parameters in each program:
a. Wave length: 120 microseconds
b. Ramp up time=1 second
c. Ramp down time=1 second
d. Stimulation time (sans ramp times)=6 seconds
e. Off/rest time=7 seconds
f. Timer, or duration of program: continuous, safety shut off at 8-10 hours of continuous use that can be restarted
   (5) FREQUENCY: Preferably five or six individual programs, each with a different frequency including but not limited to:

a. 4 Hz, 7 Hz, 12 Hz, 20 Hz, 24 Hz and 30 Hz

(6) INTENSITY: The ability to preferably vary intensity in each channel.
(7) BLUE TOOTH CONNECTIVITY: Preferably connect to an IPHONE®/ANDROID® device preferably via an app, which in turn preferably acts as a wireless control unit. The device preferably satisfies the requirements of Bluetooth wireless transmitter/receiver certifications, electrical, magnetic and wireless transmission non-interference (an FCC requirement) safety requirements, and electrical and electromagnetic safety for qualification of product liability insurance.
(8) CONTROL: Preferably wired and wireless option
a. Preferably via a separate unit which would control the stimulation unit.
b. Preferably wirelessly, via any iOS or ANDROID® compatible device.

The following is an example of the dimensions/characteristics of a control unit of a NMES device/garment of various embodiments of the present invention:

(1) SIZE: Preferably the same of smaller than the dimensions of an IPOD NANO® with buttons and screen or screen buttons only.
(2) CONNECTIVITY: Preferably wireless/a single wire to the NMES stimulator unit
(3) RECHARGING: Preferably a USB connector capable of recharging the NMES stimulator unit.
(4) PARAMETERS WOULD BE CONTROLLED IN THE NMES CONTROLLER UNIT. The following parameters would be transmitted to the STIMULATOR UNIT via BLUE TOOTH type connectivity:
a. INDIVIDUAL PROGRAMS: “P” button or touch screen selector preferably allow selection of a specific Program, each Program corresponding to a different frequency (i.e., P1=4 Hz; P2=7 Hz, etc.).
b. CHANNEL SELECTION: a button or touch screen selector that preferably allows selection of just one particular channel of the 16 channels, corresponding to a specific muscle group, which would preferably allow the “up” and “down” buttons in c) below to adjust intensity in that particular body part (since each channel corresponds to the two electrodes connected to certain body part, such as the positive and negative electrodes of the right hamstring group).
c. INTENSITY OF EACH CHANNEL—“up” and “down” buttons or touch screen selector, increasing and decreasing intensity of current in body part selected in b) above
d. BOTH CHANNELS OF BILATERALLY CORRESPONDING BODY PARTS or JUST RIGHT OR JUST LEFT:
   i. “Both” button or touch screen selector allows increase or decrease of the intensity of both channels stimulating the same corresponding body part bilaterally (i.e., both right and left hamstring group) by the “up” and “down” buttons in c);
   ii. “Right” button or touch screen selector increases or decreases the intensity of just the right body part (i.e., right hamstring group only) by the “up” and “down” buttons in c); or
   iii. “Left” button or touch screen selector increases or decreases the intensity of just the left body part (i.e., right hamstring group only) by the “up” and “down” buttons in c).

The following is an example of the dimensions/characteristics of controls of a control unit of a NMES device/garment of various embodiments of the present invention:

(1) Intensity—increase and decrease buttons or touch screen selectors;
(2) Button or touch screen selectors to change to different body parts, corresponding to different channels;
(3) Buttons or touch screen selectors to allow increase or decrease of intensity to “Both” channels controlling right and left corresponding body part (i.e., right and left hamstring, etc.), or individually control just the “R” (right) body part (hamstring) channel or just the “L” (left);
(4) “P” to select one of 5 or 6 programs which vary by frequency;
(5) On/off switch;
(6) Lock button to prevent accidental activation of other buttons or touch screen selectors; and/or
(7) Screen displaying parameters for right/left intensity for selected.

The following is an example of steps associated in developing/constructing a NMES device/garment of various embodiments of the present invention including:

Electrical Engineering:
Develop technical requirements
Select required technologies including investigation of a body network for the electrodes to reduce the wiring needs
Generate Block diagram, design summary, and high level Bill of Materials
Generate initial layout for mechanical coordination
Conduct a battery life study
Develop design options, tradeoffs, and recommendations
Develop an antenna strategy
Develop a certification strategy
Develop a requirements specification
Develop, write, and test program for controller unit
Develop interface between garment and electrodes
Develop interface electrodes and stimulator unit

Industrial Design:
Concept ideation in the form of sketches and sketch models
Preliminary CAD design of the stimulator unit housing
Investigation into an appropriate electrode lead connector component to satisfy the small-size requirements of the unit.
Concept ideation of a basic user interface

Mechanical Engineering:
Sourcing or design of an appropriate electrode lead connector component to satisfy the small-size requirements of the unit.
Engineering review/refinement of the stimulator housing design.

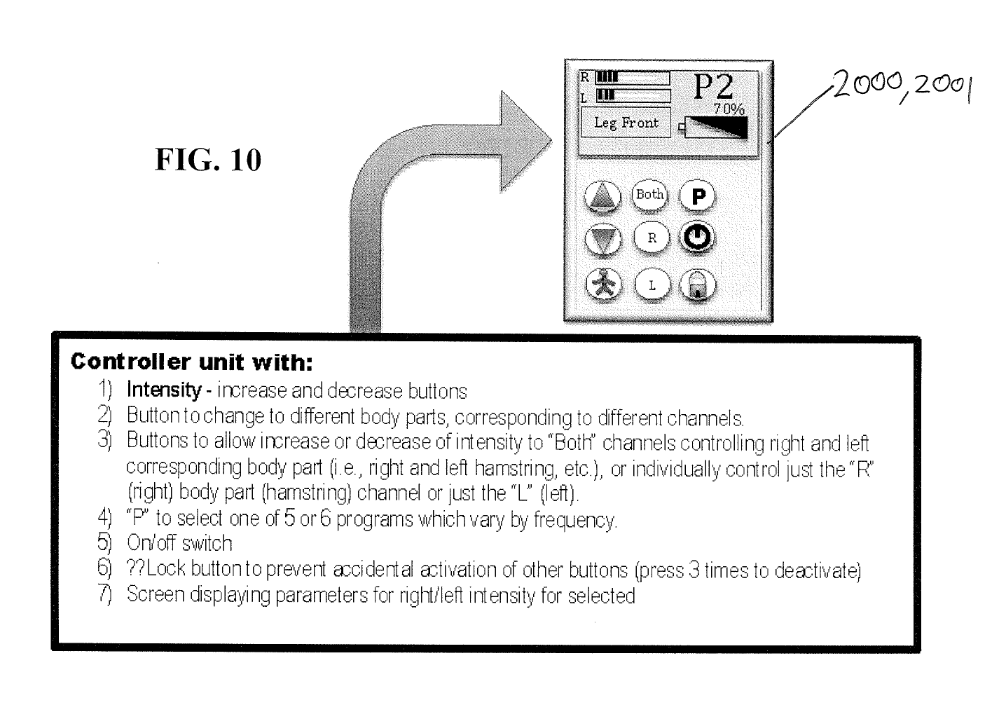

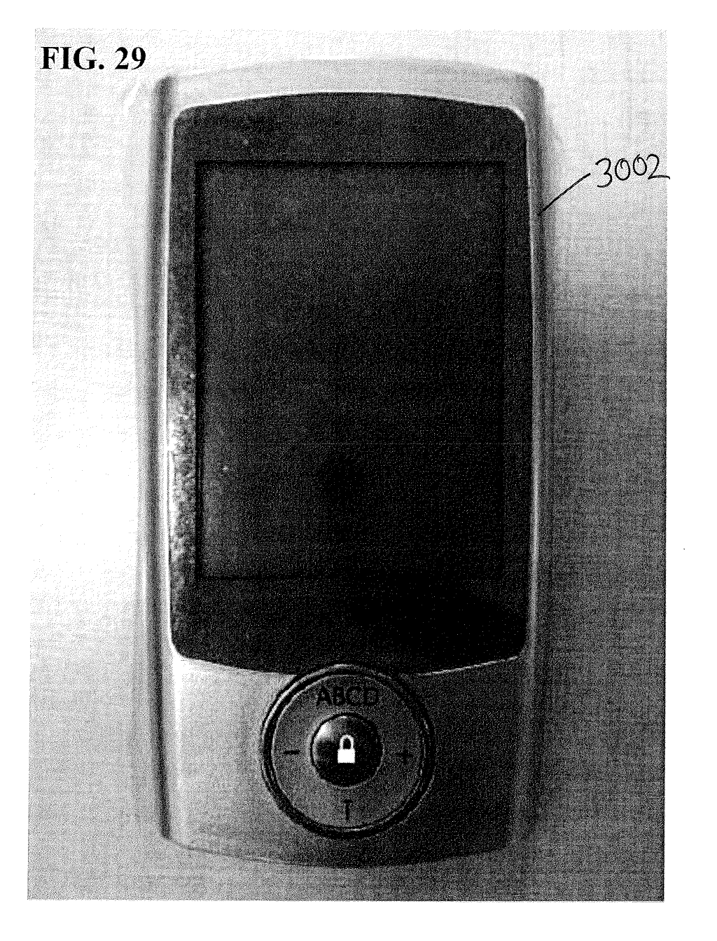

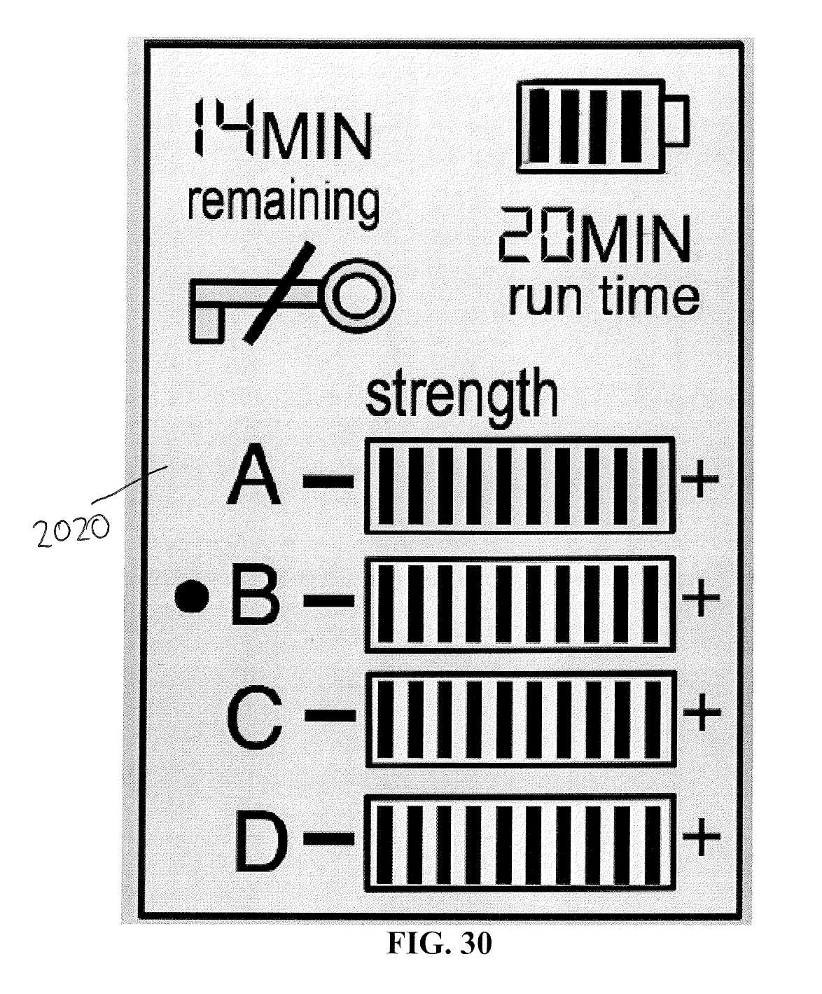

FIGS. 10-29 show examples of a user interface of various embodiments of the present invention.

Since the literature documents 24 hour stimulation periods of NMES and TENS, with no untoward side effects, we conducted a series of trials stimulating 8 large muscle groups bilaterally while wearing several versions of wearable garment to which the electrodes 500 were affixed while going about my daily activities. Our initial trial used a stimulation period of 14 hours with one side effect related to the electrodes 500 and stimulation pattern, which has now been corrected. Several repeat trials of 15½ hours resulted in no side effects. From these trials we learned a great deal, especially that significant fat loss could result from all day stimulation (although not measured directly, we estimated over 120 kcal/hr) in a device/garment combination that could be worn under normal clothing and while one goes about their daily routine. Even more importantly, we were able to determine a narrow range of optimal parameters for the NMES. We also learned what variations of garments were best tolerated, the special needs for an all-day garment based on the different comfort levels of men vs. women, special requirements for long term use electrodes 500 and that the optimal design for an NMES unit is a Bluetooth wireless unit controlled by a Bluetooth input device (smart phone 3001, tablet, smart watch, computer, etc.), which, unfortunately, is not available as an OTC product.

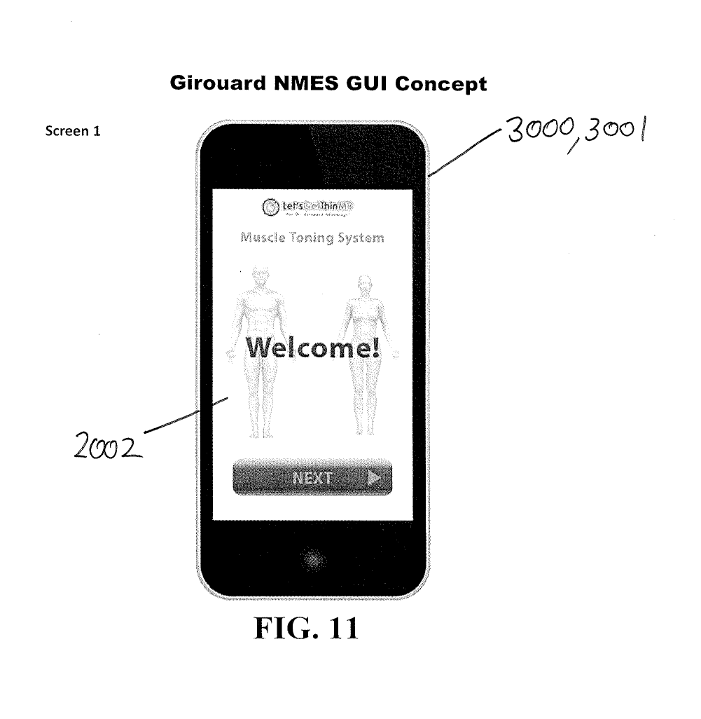

FIGS. 11-28 show a smart phone being used as a control unit of various embodiments of the present invention.

Screen 2000 includes screen 2001, 2002, 2003, 2004, 2005, 2006, 2007, 2008, 2009, 2010, 2011, 2012, 2013, 2014, 2015, 2016, 2017, 2018, 2019, and 2020.

Various embodiments as shown in FIG. 10 highlight screen 2001 showing: Controller unit with:

1) Intensity—increase and decrease buttons
2) Button to change to different body parts, corresponding to different channels.
3) Buttons to allow increase or decrease of intensity to “Both” channels controlling right and left corresponding body part (i.e., right and left hamstring, etc.), or individually control just the “R” (right) body part (hamstring) channel or just the “L” (left)
4) “P” to select one of 5 or 6 programs which vary by frequency
5) On/off switch
6) ??Lock button to prevent accidental activation of other buttons (press 3 times to deactivate)
7) Screen displaying parameters for right/left intensity for selected

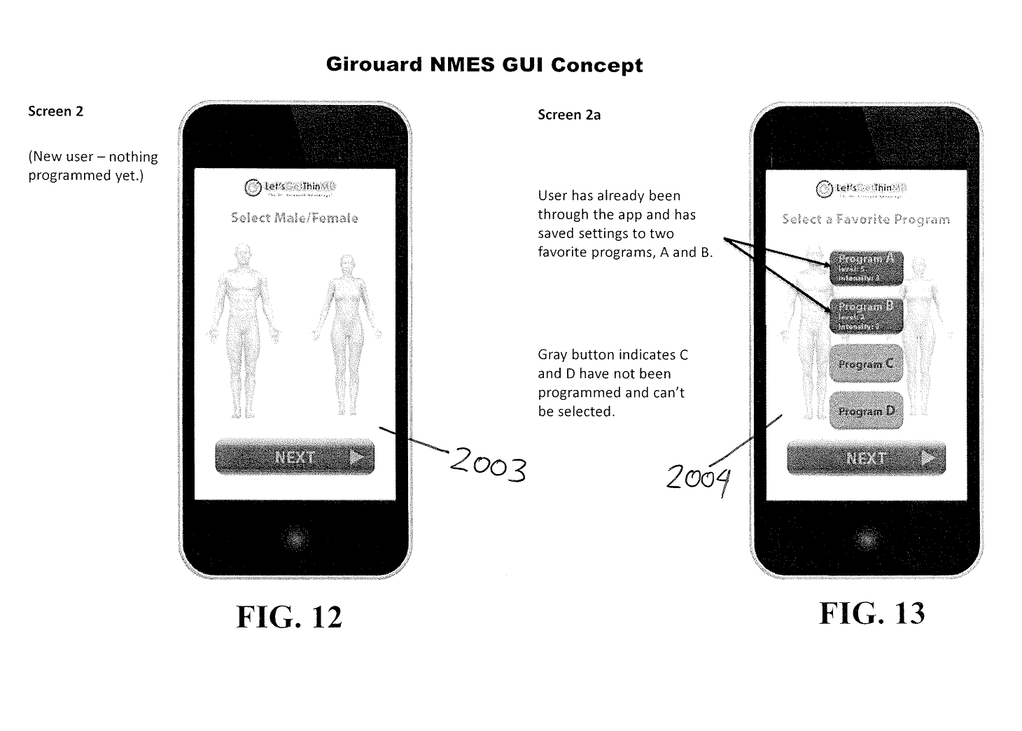

Various embodiments as shown in FIG. 12 highlight screen 2003 showing: Screen 2

### (New User—Nothing Programmed Yet.)

Various embodiments as shown in FIG. 13 highlight screen 2004 showing: Screen 2a

User has already been through the app and has saved settings to two favorite programs, A and B.
Gray button indicates C and D have not been programmed and can't be selected.

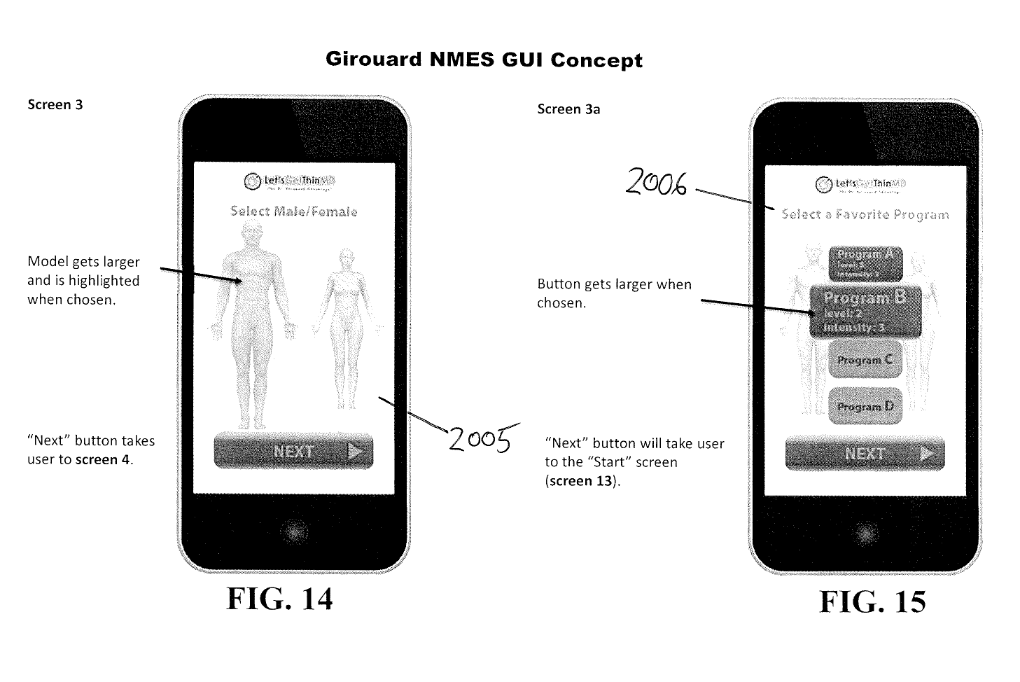

Various embodiments as shown in FIG. 14 highlight screen 2005 showing: Screen 3

Model gets larger and is highlighted when chosen.
“Next” button takes user to screen 4.

Various embodiments as shown in FIG. 15 highlight screen 2006 showing: Screen 3a

Button gets larger when chosen.
“Next” button will take user to the “Start” screen (screen 13).

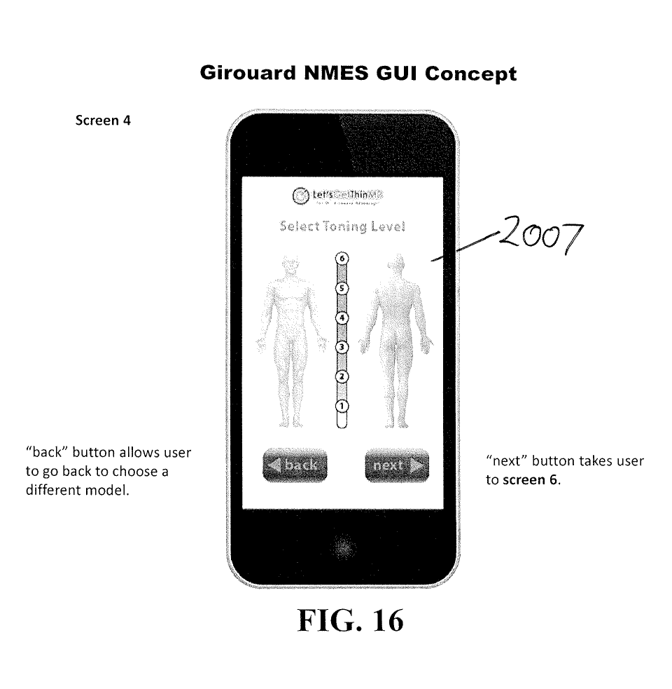

Various embodiments as shown in FIG. 16 highlight screen 2007 showing: Screen 4

“back” button allows user to go back to choose a different model.
“next” button takes user to screen 6.

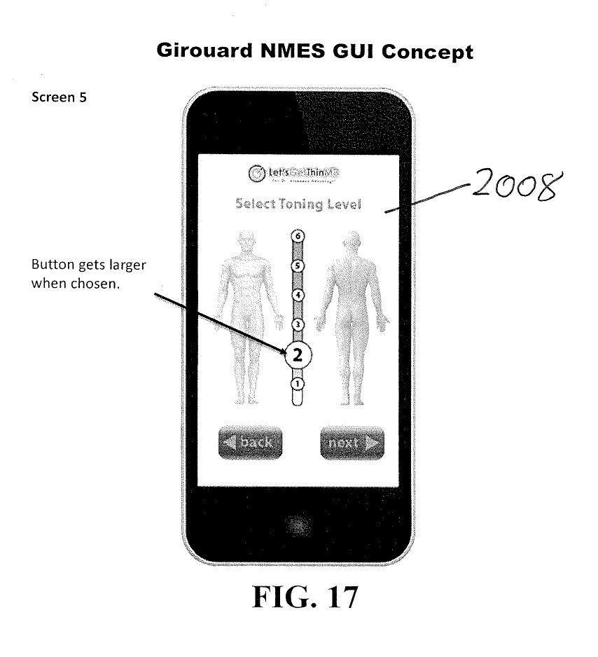

Various embodiments as shown in FIG. 17 highlight screen 2008 showing: Screen 5

Button gets larger when chosen.

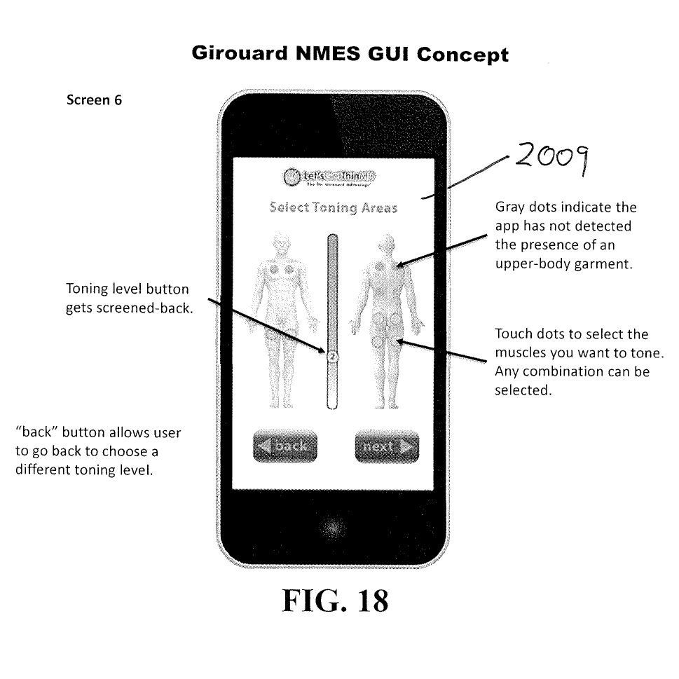

Various embodiments as shown in FIG. 18 highlight screen 2009 showing: Screen 6

Toning level button gets screened-back.
“back” button allows user to go back to choose a different toning level.
Gray dots indicate the app has not detected the presence of an upper-body garment.
Touch dots to select the muscles you want to tone. Any combination can be selected.

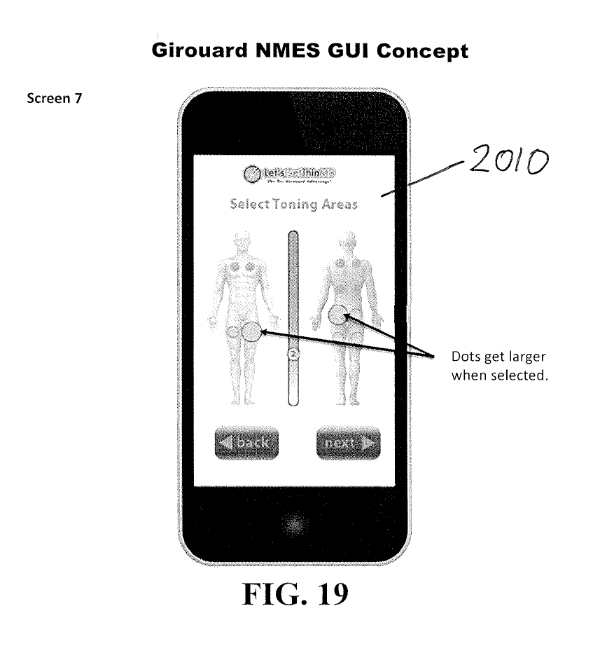

Various embodiments as shown in FIG. 19 highlight screen 2010 showing: Screen 7

Dots get larger when selected.

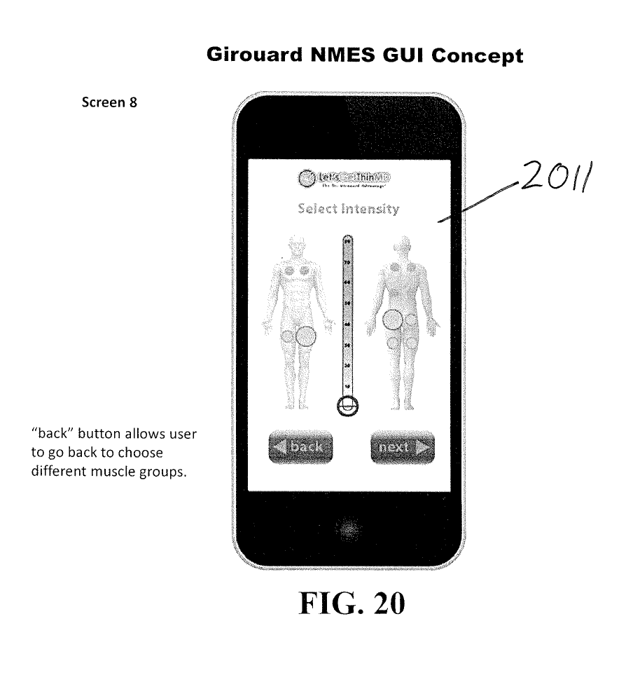

Various embodiments as shown in FIG. 20 highlight screen 2011 showing: Screen 8

“back” button allows user to go back to choose different muscle groups.

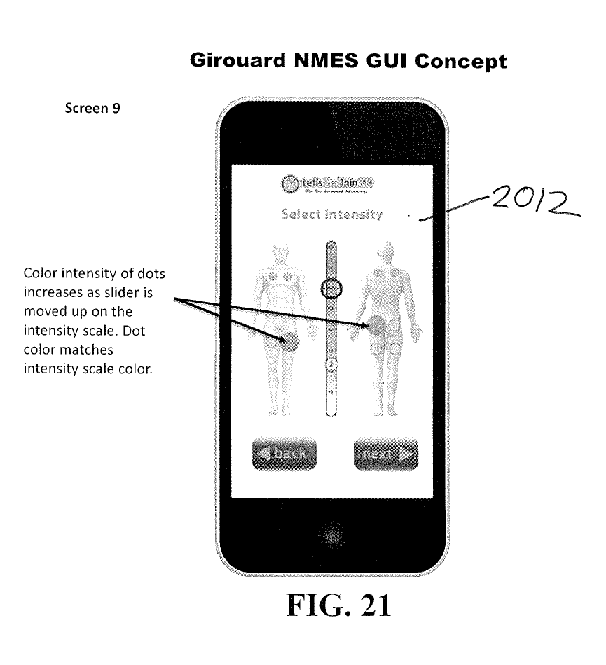

Various embodiments as shown in FIG. 21 highlight screen 2012 showing: Screen 9

Color intensity of dots increases as slider is moved up on the intensity scale. Dot color matches intensity scale color.

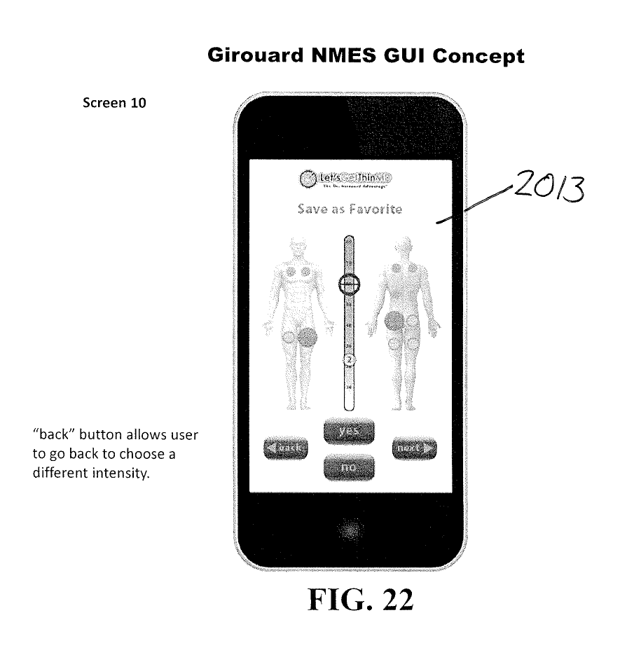

Various embodiments as shown in FIG. 22 highlight screen 2013 showing: Screen 10

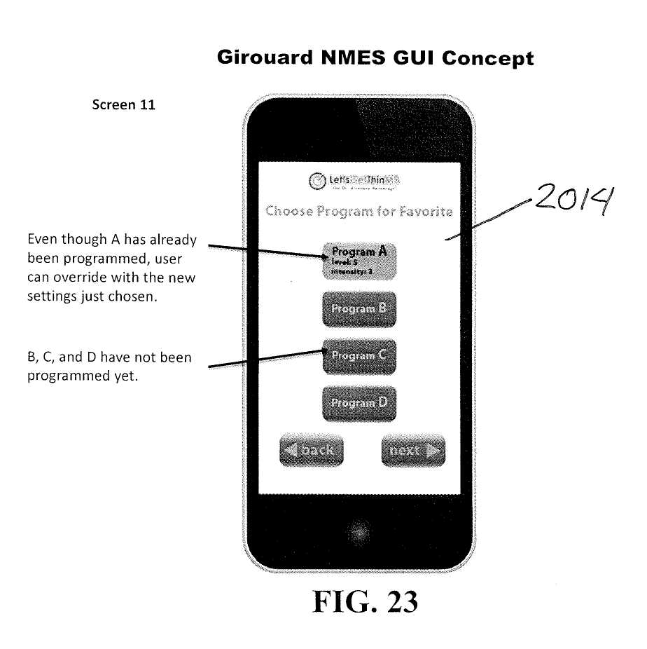

“back” button allows user to go back to choose a different intensity.
Various embodiments as shown in FIG. 23 highlight screen 2014 showing: Screen 11
Even though A has already been programmed, user can override with the new settings just chosen.
B, C, and D have not been programmed yet.

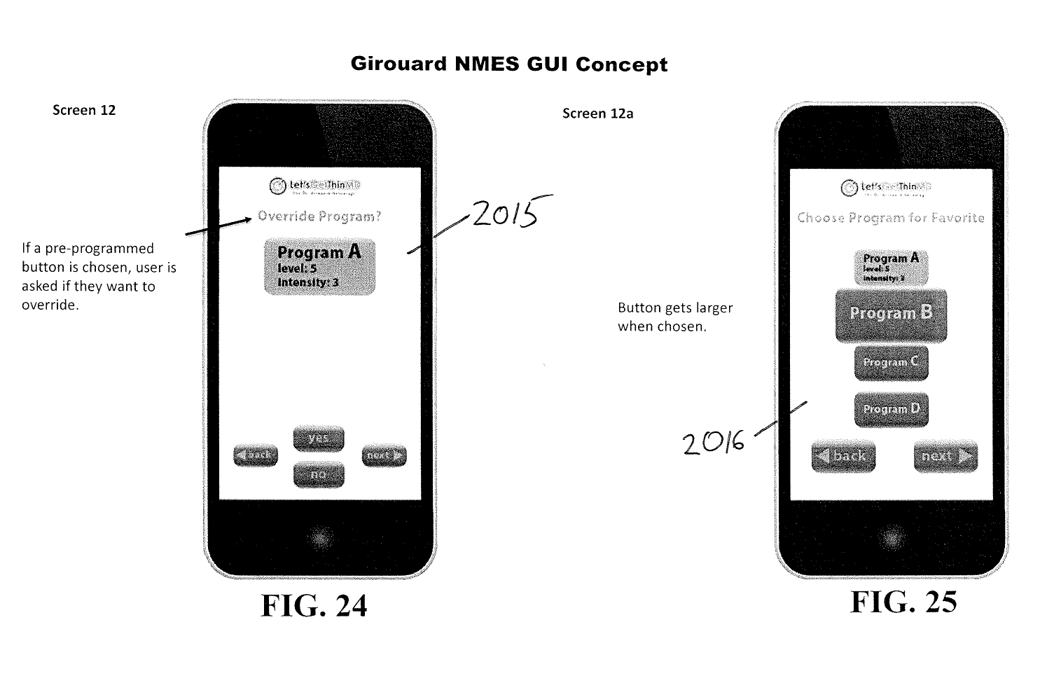

Various embodiments as shown in FIG. 24 highlight screen 2015 showing: Screen 12

If a pre-programmed button is chosen, user is asked if they want to override.

Various embodiments as shown in FIG. 25 highlight screen 2016 showing: Screen 12a

Button gets larger when chosen.

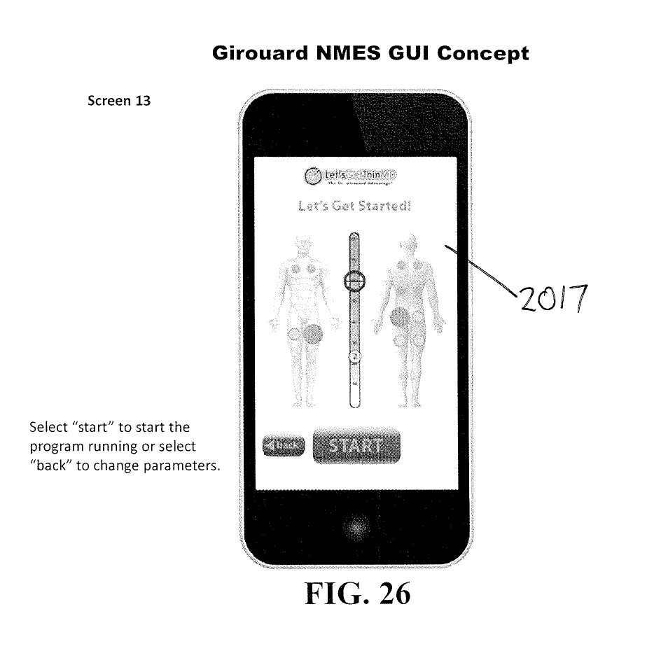

Various embodiments as shown in FIG. 26 highlight screen 2017 showing: Screen 13

Select “start” to start the program running or select “back” to change parameters.

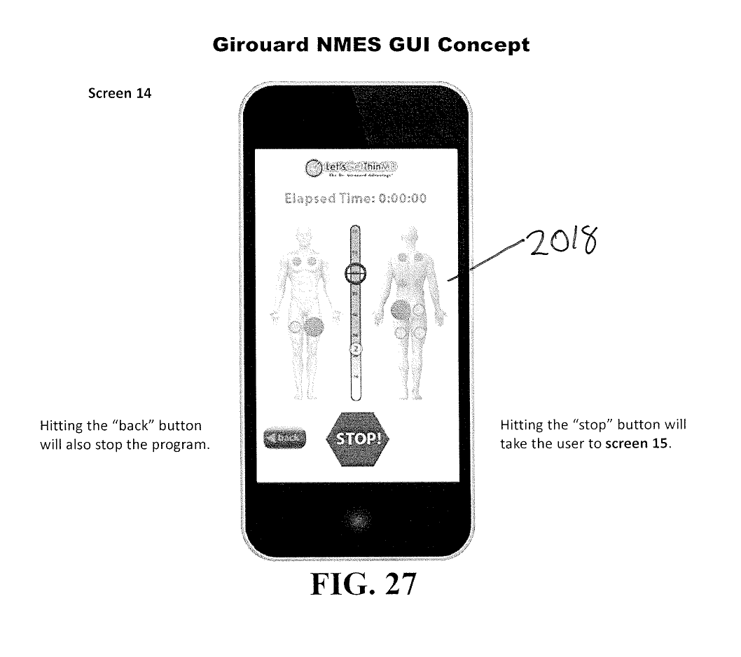

Various embodiments as shown in FIG. 27 highlight screen 2018 showing: Screen 14

Hitting the “back” button will also stop the program.
Hitting the “stop” button will take the user to screen 15.

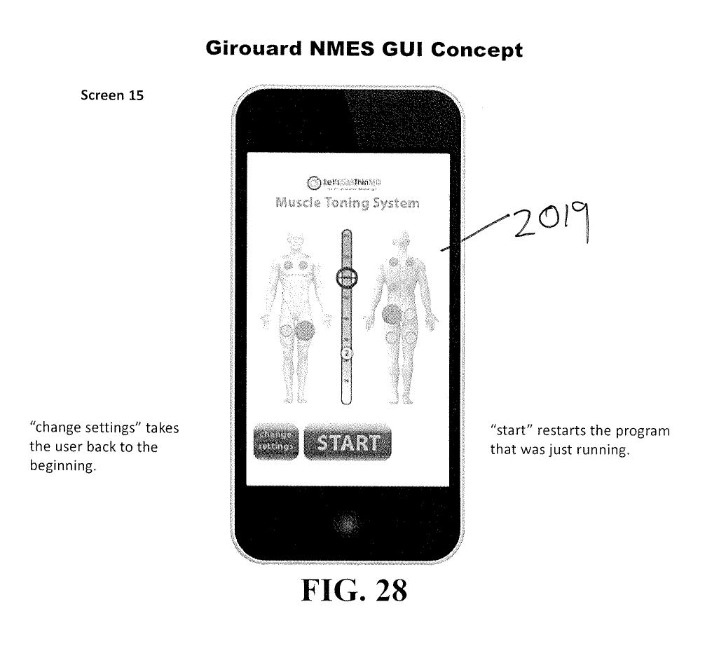

Various embodiments as shown in FIG. 28 highlight screen 2019 showing: Screen 15

“change settings” takes the user back to the beginning.
“start” restarts the program that was just running.

The apparatus of any embodiment of the present invention is unique when compared to other devices already marketed, such as the German “Miah” and the Turkish “X-body”. These suits produce high intensity neuromuscular stimulation in opposed muscle groups, are not portable, have exposed wires and bulky suits and are used in conjunction with either cross training or weight training exercises to increase muscle tone, muscle mass and calorie consumption. Because of the specific parameters of the NMES (frequency, intensity, stimulation pattern, wave length, etc.), these devices mainly stimulate Type 2, slow twitch muscle fibers which primarily burn glucose (or glycogen)—not fat—for energy. Furthermore, with continued use over time, Type 1 slow twitch muscle fibers which burn fat instead of glucose are converted into Type 2 fibers, reducing the fat burning ability of an individual. On the other hand, low intensity, prolonged stimulation with the parameters we've found are optimal for fat burning provides optimal fat tissue metabolism and fat tissue loss.

In various embodiments, the NMES device/garment comprises a garment made of a flexible material and electrodes 500, wiring, and NMES unit(s) that are preferably and removably attached to the garment

In various embodiments, the apparatus 100 comprises a means for attaching electrodes 500, wiring, and NMES unit(s) to the garment for muscular stimulation. In various embodiments, the means allow for the electrodes 500, wiring, and NMES unit(s) to be affixed to the garment at various positions.

In various embodiments, the electrodes 500, wiring, and NMES unit(s) are contained within compartments 400 such as bags having an outer surface that can be removably attached to the inner surface of the garment.

In various embodiments, the compartments 400 containing the electrodes 500 have opening allowing for the electrodes 500 to be positioned on the skin of a user/wearer. In various embodiments, the wiring is placed within compartments 400 such as within the bore/channel of tubing such that the compartments 400 are removably attached to the garment.

In various embodiments, the garments have channels through which the wires can be removable placed within.

In various embodiments, the electrodes 500, wiring, and NMES unit(s) directly and removably attach to the garment. The electrodes 500, wiring, and NMES unit(s) of various embodiments may have a surface with a means for directly attaching the garment.

In various embodiments, the attachment means is a hook and loop fastener 900 (i.e., VELCRO®) with the loops preferably on the garment and hooks on the electrodes 500, wiring, and NMES unit(s).

In various embodiments, the attachment means is a hook and loop fastener 900 (i.e., VELCRO®) with the loops preferably on the electrodes 500, wiring, and NMES unit(s) and hooks on the garment.

In various embodiments, the apparatus 100 includes a means for attaching/securing the upper and lower sections of the garment together.

In various embodiments, the means for attaching/securing the upper and lower sections is a zipper.

In various embodiments, the means for attaching/securing the upper and lower sections is a hook and loop fastener 900 (i.e., VELCRO®).

In various embodiment, the wires for conducting an electrical pulse to the electrodes 500 preferably run along channels within the garment.

In various embodiments, the hook and loop fastener 900 of any embodiment are preferably spaced a part strips to allow for bending easily when sitting.

In various embodiments, the electrodes 500 are either FDA approved sticky or water activated cloth weave.

In various embodiments, the upper section of the apparatus 100 of any embodiment of the present invention has 1, 2, 3, 4, 5, 6, 7, 8, 9, 10, 11, 12, 13, 14, 15, 16, 17, 18, 19, and 20 electrodes. In various embodiments, the number of electrodes in the upper section falls within the range between any two of the above numbers.

In various embodiments, the upper section of the apparatus 100 of any embodiment of the present invention has 1, 2, 3, 4, 5, 6, 7, 8, 9, and 10 NMES unit(s). In various embodiments, the number of NMES unit(s) in the upper section falls within the range between any two of the above numbers.

In various embodiments, the lower section of the apparatus 100 of any embodiment of the present invention has 1, 2, 3, 4, 5, 6, 7, 8, 9, 10, 11, 12, 13, 14, 15, 16, 17, 18, 19, and 20 electrodes. In various embodiments, the number of electrodes in the lower section falls within the range between any two of the above numbers.

In various embodiments, the lower section of the apparatus 100 of any embodiment of the present invention has 1, 2, 3, 4, 5, 6, 7, 8, 9, and 10 NMES unit(s). In various embodiments, the number of NMES unit(s) in the lower section falls within the range between any two of the above numbers.

In various embodiments, the wires from the stimulator unit/NMES unit preferably run down the outside of the lateral strip in a tunnel or channel preferably made of sewn cloth and preferably connect to the end connectors of the electrode wires preferably protruding through a hole preferably in the leg band. The wires of any embodiment of the present invention may be of variable thickness. In various embodiments, the connectors are a male to female 2 mm pin that preferably slides tightly in. In alternative embodiments, the connectors are preferably snaps. The embodiments of containment of the wires, preferably eliminate the need to connect each electrode to its wire from the stimulator unit/NMES machine, and the easy quick placement of the electrodes preferably and greatly reduces the time spent setting up the apparatus 100 and preferably aids in preventing wire entanglement when the apparatus is removed.

In various embodiments, the wire connections are preferable accomplished through a to 3.5 mm plug.

In various embodiments, electrode(s) is/are preferably positioned in a manner to preferably stimulate abdominal muscles.

In various embodiments, the electrode(s) is/are preferably located on the inner surface of apparatus 100.

In various embodiments, electrodes in the lower section are preferably positioned below the belly button of a wearer.

In various embodiments, the apparatus 100 further comprises electrode(s) in the upper section preferably positioned below the rib cage of a wearer. The electrode(s) may include a complimentary set of electrodes positioned above the electrode(s) positioned below the belly button and below the rib cage. The electrode(s) are preferably attached to the upper section of the garment.

In various embodiments, the apparatus 100 further comprises electrode(s) removably attached to the lower section and preferably positioned to upper and lower ends of both gluteal muscles.

In various embodiments, placement of electrodes for the upper and lower section are the same for the one piece garment.

In various embodiments, the apparatus 100 comprises electrodes preferably positioned to upper and lower ends of bilateral gluteal muscles, upper and lower ends of bilateral hamstrings, upper and lower ends of bilateral quadriceps, upper and lower ends of the gastrocnemius muscle, and upper and lower ends of bilateral abdominal muscles. In this embodiment, the apparatus 100 comprises a section of material preferably made of neoprene and is similar to the lower aspect of a wetsuit type of garment.

In various embodiments, the apparatus 100 comprises electrodes preferably positioned to upper and lower ends of bilateral latissimus dorsi muscles, upper and lower ends of bilateral trapezius muscles, upper and lower ends of bilateral pectoralis major muscles, and upper and lower ends of bilateral biceps muscle. In this embodiment, the apparatus 100 comprises a section of material preferably made of neoprene and is similar to the lower aspect of a wetsuit type of garment.

The following are examples of embodiments of the present invention.

The stimulator/electrical generating unit of any embodiment of the present invention preferably has 4 to 16 channels. More preferably, the stimulator/electrical generating unit of any embodiment of the present invention has 4 to 10 channels. Most preferably, the stimulator/electrical generating unit of any embodiment of the present invention has 8 to 10 channels.

The stimulator/electrical generating unit of any embodiment of the present invention preferably has 1 to 10 programs. More preferably, the stimulator/electrical generating unit of any embodiment of the present invention has 1 to 8 programs. Most preferably, the stimulator/electrical generating unit of any embodiment of the present invention has 1 to 4 programs.

In various embodiments, the stimulator/electrical generating unit such as, for example, SM9028T 3002, SM9028 NT 3002, and SM9028NE 3002 units preferably facilitate 4 different NMES programs and preferably include a user interface. FIG. 32 shows an example of a user interface of various embodiments of the present invention.

Each program preferably has the same parameters, but a different NMES frequency. In various embodiments, existing units are reprogrammed to preferably facilitate 4 different NMES programs. In alternative embodiments, the stimulator/electrical generating unit facilitates 5 different NMES programs.

In various embodiments, the units are preferably worn on a belt clip.

In various embodiments, Program 1 is 4 Hz.

In various embodiments, Program 2 is 7 Hz.

In various embodiments, Program 3 is 12 Hz.

In various embodiments, Program 4 is 18 Hz.

In various embodiments, the parameters for each of the NMES programs are preferably as follows:

1. pulse shape=biphasic symmetric square wave.

Other types of pulse shapes that may be used for any embodiment of the present invention include but are not limited to biphasic symmetric square wave, biphasic assymmetric wave, biphasic assymmetric square wave, and biphasic assymmetric wave.

2. pulse width=120 milliseconds.

The pulse width used for any embodiment of the present invention preferably ranges from 50 microseconds to 300 microseconds. More preferably, the pulse width used for any embodiment of the present invention ranges from 70 microseconds to 210 microseconds. Most preferably, the pulse width used for any embodiment of the present invention ranges from 120 microseconds to 140 microseconds.

3. ramp up time=1 second

The ramp up time used for any embodiment of the present invention preferably ranges from 0.5 second(s) to 7 second(s). More preferably, the ramp up time used for any embodiment of the present invention ranges from 1 second(s) to 3 second(s). Most preferably, the ramp up time used for any embodiment of the present invention ranges from 1 second(s) to 2 second(s).

4. ramp down time=1 second

The ramp down time used for any embodiment of the present invention preferably ranges from 0.5 second(s) to 7 second(s). More preferably, the ramp down time used for any embodiment of the present invention ranges from 1 second(s) to 3 second(s). Most preferably, the ramp down time used for any embodiment of the present invention ranges from 1 second(s) to 2 second(s).

5. stimulation time (preferably excluding the ramp up & ramp down times)=6 seconds

The stimulation time (preferably excluding the ramp up & ramp down times) used for any embodiment of the present invention preferably ranges from 2 second(s) to 21 second(s). More preferably, the stimulation time (preferably excluding the ramp up & ramp down times) used for any embodiment of the present invention ranges from 3 second(s) to 12 second(s). Most preferably, the stimulation time (preferably excluding the ramp up & ramp down times) used for any embodiment of the present invention ranges from 6 second(s) to 12 second(s).

6. off time or rest time with no stimulation=7 seconds

The off time or rest time with no stimulation used for any embodiment of the present invention preferably ranges from 1 second(s) to 21 second(s). More preferably, the off time or rest time with no stimulation used for any embodiment of the present invention ranges from 3 second(s) to 18 second(s). Most preferably, the off time used or rest time with no stimulation for any embodiment of the present invention ranges from 6 second(s) to 12 second(s).

7. a timer function that preferably allows for continuous stimulation for up to 12-14 hours.

The timer function used for any embodiment of the present invention preferably ranges from 10 minutes to 24 hour(s). More preferably, the timer function used for any embodiment of the present invention ranges from 15 minutes to 20 hour(s). Most preferably, the timer function used for any embodiment of the present invention ranges from 15 minutes to 16 hour(s).

Alternatively, the timer function used for any embodiment of the present invention preferably ranges from 10 minutes to 24 hour(s). More preferably, the timer function used for any embodiment of the present invention ranges from 15 minutes to 20 hour(s). Most preferably, the timer function used for any embodiment of the present invention ranges from 15 minutes to 16 hour(s).

In various embodiments, the timer preferably increases exercise time gradually and does not reset the apparatus/device 100 for the entire stimulation time. For example, the timer may increases exercise time gradually to an 8 hour routine. The timer allows for different stimulation times including but not limited to, for example, 4 hours of stimulation, 8 hours of stimulation, and 12 hours of stimulation. Alternatively for example, the timer may allow for 1 hour increments including 1 hour, 2 hours, 3 hours, 4 hours, 5 hours, 6 hours, 7 hours, or 8 hours options or half hour increment options such as 3.5 hours, 6.0 hours, or 4.5 hours. In another embodiment, the timer may be set up for increments up to eight hours and may allow for 20 minute increments.

In another embodiment, the timer for stimulation time is preferably adapted for battery life and health consideration and includes but is not limited to stimulation times ranges from 10 minutes to 60 minutes. In this embodiment, 20 minutes is the default stimulation time and the timer also includes stimulation time options for 10 minutes, 30 minutes, 40 minutes, 50 minutes, and 60 minutes.

In various embodiments, the NMES unit/the stimulator/electrical generating unit is preferably retooled to contain four channels. In another embodiment, the unit contains eleven channels. In another embodiment, the unit contains twelve channels.

In various embodiments, the channels preferably allow for independent intensity adjustment. In various embodiments, the control unit preferably controls intensity adjustment in the NMES unit/the stimulator/electrical generating unit.

In various embodiments, the user interface of NMES unit/the stimulator/electrical generating unit preferably includes a pair of buttons for each channel to increase and decrease intensity. For example, the unit preferably has “+” and “−” buttons for channels labeled “A”, “B”, “C” and “D”.

In alternative embodiments, the user interface preferably includes one button allowing for a user to scroll through the different intensities. In this embodiment, using one button preferably allows for the unit to be of a smaller size and cost effective.

In various embodiments, the NMES unit/the stimulator/electrical generating unit preferably includes a screen lock.

In various embodiments, the NMES unit/the stimulator/electrical generating unit is preferably a Bluetooth NMES device with 8-12 channels supporting 16-24 electrodes for 12-14 hours, rechargeable via USB and as small as possible.

In various embodiments, the NMES unit/the stimulator/electrical generating unit preferably has a small, cell phone sized design. More preferably, small, cell phone sized design includes means allowing for a user/wearer to separately control each channel's intensity.

The software used for any embodiment of the present invention includes but is not limited to, for example, Android, OSX, Microsoft and iOS platforms.

In various embodiments, the NMES unit/the stimulator/electrical generating unit is a “off the shelf” NMES units that has been preferably modified. More preferably, the “off the shelf” NMES units has FDA approval/clearance.

In various embodiments, the design of the NMES unit/the stimulator/electrical generating unit is preferably a modification of an existing FDA approved device and duplicates parameters already used by other FDA approved NMES devices.

In various embodiments, the channels are preferably adjustable for intensity independent. In another embodiment, 12 channels are preferably adjustable for intensity independent to preferably comply with regulations related to a transmitter/receiver device.

In various embodiments, the NMES unit/the stimulator/electrical generating unit is a non-BLUETOOTH® NMES having 4 channels.

In various embodiments, the modification of an existing schematic design of the NMES unit/the stimulator/electrical generating unit preferably allows for a user/wearer to change the intensity of each channel independent of the other channels. For example, modification of existing schematic design of an existing schematic design of the NMES unit/the stimulator/electrical generating unit having 12 channels allows a user/wearer to change the intensity of one channel independent of the other 11 channels.

Various embodiments of the present invention includes plans, for example, for: manufacturing and assembly of NMES the NMES unit/the stimulator/electrical generating unit; assembly of components of the suit such as NMES unit, wiring, electrodes; or assisting with packaging or manual.

In various embodiments, the present invention preferably includes a BLUETOOTH® Device(s) for controlling the NMES unit where the BLUETOOTH® Device(s) preferably includes software for implementing control over the NMES. The software is preferably developed from popular languages including but not limited to iOS, Android, or Microsoft.

In various embodiments, the other settings for electrical muscular stimulation (EMS) are preferably the same for all frequencies for the four channels and are preferably as follows:

1. pulse shape=biphasic symmetric square wave

2. pulse width=120 milliseconds

3. ramp time=1 second

4. ramp down time=1 second

5. stimulation time (preferably excluding the ramp up & ramp down times)=6 seconds

6. off time=7 seconds

7. a timer function that preferably allows for continuous stimulation for up to 12-14 hrs.

In various embodiments, the stimulator/electrical generating unit facilitates 5 different NMES programs preferably allowing 5 different EMS frequencies including, for example, 4 Hz, 7 Hz, 12 Hz, 20 Hz and 30 Hz. In this embodiment, the other settings for the EMS are preferably the same for all 5 frequencies and are, for example, as follows: pulse shape=biphasic symmetric square wave; pulse width=120 milliseconds; ramp up time=1 second; ramp down time=1 second; stimulation time (preferably excluding the ramp up & ramp down times)=6 seconds; off time=7 seconds. In this embodiment, the stimulator/electrical generating unit preferably includes a time function that preferably allows for continuous stimulation such as, for example, low intensity stimulation patterns preferably for up to 16 hours.

In various embodiments, the stimulator/electrical generating unit preferably includes 4 channels, 5 different programs and preferably allows 5 different EMS frequencies, and other parameters.

In various embodiments, the stimulator/electrical generating unit is preferably a SM9028NT with 4 channels with massage modes and intensity adjusted.

In various embodiments, the stimulator/electrical generating unit is preferably a SM9028NT with 4 channels with massage modes, intensity adjusted, tooling shell, and printable circuit board (PCB).

In various embodiments, the stimulator/electrical generating unit is preferably of a small size and has the ability to change intensity on each channel.

In various embodiments, the stimulator/electrical generating unit preferably has four channels that preferably stimulates eight electrodes. In this embodiment, an external splitter such as a Y connector is preferably plugged into each of the two channels on the unit and preferably uses an RCA connecter.

In various embodiments, a koalaty connector is used.

In various embodiments, the stimulator/electrical generating unit preferably includes means for adjusting the intensity on all channels.

In various embodiments, the stimulator/electrical generating unit is a SM9128 unit that may be modified. See http://www.sunmas.hk/products-detail.php?ProId=49&PHPSESSID=8a773905dbb84770905b079d73804ba5

In various embodiments, the stimulator/electrical generating unit is a modified SM9028NT unit. The modified SM9028NT unit preferably includes 8 massage modes and 2 independent channels and preferably has the same dimensions as the SM9128 unit.

In various embodiments, the stimulator/electrical generating unit is an FDA approved/cleared unit.

In various embodiments, the stimulator/electrical generating unit is an FDA 510(K) approved/cleared unit.

In various embodiments, the stimulator/electrical generating unit that is an FDA approved/cleared or an FDA 510(K) approved/cleared unit includes devices such as but not limited to stimulators, massagers, or different products having a Device Listing Number and/or an FDA Product Code.

In various embodiments, the stimulator/electrical generating unit has an acceptable failure rate.

In various embodiments, the electrodes are various silicone electrodes including silicone pads. The silicon electrodes are preferably used with sponge, preferably for water absorption, and a bandage, preferably used for tying up the pads to an individual's body.

In various embodiments, the electrodes are various silver & carbon fiber layer electrodes. The silver & carbon fiber layer electrodes are preferably, but not necessarily, used with some type of conductive material, such as conductive gel, water or perspiration. The electrode has a hook-type (i.e., Velcro) backing.

In another embodiment, the electrodes are composed of conductive knitted fabric, such as silver fiber, with a hook-type (i.e., Velcro) backing.

In another embodiment, the electrodes are composed of silver film weave with carbon fiber backing, with a hook-type (i.e., Velcro) backing.

Other types of electrodes that may be used for any embodiment of the present invention, for example, include but are not limited to soft silicon electrodes with a carbon or silver material or other type of conducting surface, self adhesive electrodes with a carbon or silver material or other type of conducting surface.

In various embodiments, the electrodes are 7 cm×11 cm. In another embodiment, the electrodes 1.875 inch (4.76 cm) round electrodes. In another embodiment, the electrodes are 2.5 inch (6.35 cm) round electrodes. In another embodiment, various size and shape electrodes (round, oval, square, rectangular) are used as best suits the individual.

The electrodes used for any embodiment of the present invention preferably have a length ranging from 4.76 cm to 12 cm. More preferably, the electrodes used for any embodiment of the present invention have a length ranging from 4.76 cm to 10 cm. Most preferably, the electrodes used for any embodiment of the present invention have a length ranging from 4.76 cm to 8 cm.

The electrodes used for any embodiment of the present invention preferably have a width ranging from 4.76 cm to 12 cm. More preferably, the electrodes used for any embodiment of the present invention have a width ranging 4.76 cm to 10 cm. Most preferably, the electrodes used for any embodiment of the present invention have a width ranging from 4.76 cm to 8 cm.

In various embodiments, the silicone electrodes are preferably connected to the skin made more conductive with the addition of a fluid. The fluid is preferably water.

In various embodiments, the silver cloth electrodes are made more conductive with the addition of a fluid. The fluid is preferably water.

In various embodiments, the silicone electrodes are made more conductive with the addition of conducting gel.

In various embodiments, the silver cloth electrodes are made more conductive with the addition of conducting gel.

In various embodiments, the electrodes are preferably made of or include a hypoallergenic sticky material. Hypoallergenic materials used for any embodiment of the present invention, for example, include any currently FDA approved, non-latex material. In various embodiments, hypoallergenic materials includes materials that have received prior 510(k) marketing clearance with a claim of hypoallergenicity.

In various embodiments, the electrodes are black and silver square type silver thread or cloth attached to a non-conductive cloth material that are preferably activated when wet. In various embodiments, the electrodes are silver thread or cloth attached to a non-conductive cloth material that are made more conductive when wet.

In other embodiments, the electrodes are made up of a highly conductive silver and carbon fiber affixed to a non-conductive support material (silicon, plastic, etc.), made more conductive by the body's natural perspiration, conducting gel or water.

In various embodiments, the electrodes preferably include pin connectors preferably at an end of the electrodes. The pin connectors are preferably small and stay securely in the machine. In various embodiments, the pin connectors are preferably standard pin connectors. In alternative embodiments, the pin connectors are preferably snap connecters. In alternative embodiments, the pin connectors are koalaty connecters.

Other types of connectors that may be used with the device for any embodiment of the present invention, for example, include Molex connectors.

In various embodiments, the device includes various wire length options.

The wires preferably used for any embodiment of the present invention have a length ranging from 20 cm to 120 cm. More preferably, the wires used for any embodiment of the present invention have a length ranging from 30 cm to 100 cm. Most preferably, the wires used for any embodiment of the present invention have a length ranging from 40 cm to 90 cm.

In various embodiments, the device further comprises a means for adjusting wire length.

In various embodiments, the device includes wires approved for use with FDA approved/cleared devices. The wires approved for use with FDA approved/cleared devices preferably have the following characteristics: Φ2.3 C; Plug 13 mm; 150 cm length; Pull Resistance of 1.8; Connection: Pin connection (1*2, one connection two pins); and/or made up of Tinsel Material.

In various embodiments, the device includes a housing. The housing preferably protects the buttons from being accidentally pushed. In various embodiments, the housing is preferably a plastic hollow tube. More preferably, the housing is preferably a simple plastic hollow tube. The housing may be made of other materials including, for example, latex, latex free material, silicone, etc. Examples include a thin exercise band tube, surgical tubing or Penrose drain.

In various embodiments, the device further comprises a means for running wires and storing wires and electrodes. The means for running wires is preferably a secondary garment, such as a bag for example, that is preferably thin and is preferably removably affixed to the garment. The secondary garment is preferably able to store the wires and electrodes. The secondary garment is preferably removably affixed to the garment by a means such as but not limited to a hook and loop fastener 900 (i.e., VELCRO®) and may be positioned on the garment in a manner including but not limited to inside of or on the outside of the garment. The secondary garment with wires and electrodes are preferably detached and affixed to the garment in a one step process. The secondary garment may be made of other materials including, for example, Gortex, nylon, neoprene, latex, etc. In these embodiments, the garment may be made up of a lighter, breathable, and cooler material than neoprene such as but not limited to spandex or spandex composite and/or may be made of a material that is washable. The garment may be made of other materials including, for example, spandex, spandex blend, double knits, etc. The garment may also be odor proof and the use of the lighter (i.e. lightweight, lower weight, etc.), breathable (i.e absorbent), and cooler material (i.e. temperature regulating) preferably extends the odor resistance for a longer period of time and preferably extends the lifetime of the garment's use. For example, material that is odor proof maintains this odor resistance for 50 washings. This may still require 6 suits for a year, which may be desirable. Since neoprene suit would likely need to be replaced after a 12-18 months of use, multiple suits may be a good option. Also, the lighter, breathable, and cooler material is preferable for use in warmer temperatures and is preferably less expensive than other comparable materials.

In various embodiments, garment is made up of a soft, four way super stretch material. In this embodiment, the garment is preferably modified with zippers and has a bigger cut out in perineal area.

In various embodiments, the device includes Penrose drain tubes with a hook and loop fastener 900 (i.e., VELCRO®) attached.

### LIST OF REFERENCE NUMERALS

The following is a list of reference numerals used in this specification:

| Reference Numeral | Description |
|---|---|
| 100 | Apparatus/Device |
| 200 | Garment |
| 210 | Upper Section |
| 220 | Lower Section |
| 230 | Crotch Cut Out/Opening in the Crotch Area |
| 240 | Zipper |
| 250 | Zipper |
| 260 | Zipper |
| 300 | NMES/Stimulator Unit |
| 400 | Compartment/Bag |
| 401 | Compartment/Bag |
| 402 | Compartment/Bag |
| 403 | Compartment/Bag |
| 404 | Compartment/Bag |
| 405 | Compartment/Bag |
| 406 | Compartment/Bag |
| 407 | Compartment/Bag |
| 408 | Compartment/Bag |
| 409 | Compartment/Bag |
| 410 | Compartment/Bag |
| 411 | Compartment/Bag |
| 412 | Compartment/Bag |
| 413 | Compartment/Bag |
| 414 | Compartment/Bag |
| 415 | Compartment/Bag |
| 500 | Electrode |
| 600 | Wire |
| 601 | Wire |
| 602 | Wire |
| 603 | Wire |
| 604 | Wire |
| 605 | Wire |
| 606 | Wire |
| 607 | Wire |
| 608 | Wire |
| 700 | Channel |
| 701 | Channel |
| 702 | Channel |
| 703 | Channel |
| 704 | Channel |
| 705 | Channel |
| 800 | Sheet |
| 801 | Sheet |
| 802 | Sheet |
| 803 | Sheet |
| 804 | Sheet |
| 805 | Sheet |
| 806 | Sheet |
| 807 | Sheet |
| 808 | Sheet |
| 809 | Sheet |
| 810 | Sheet |
| 900 | Hook and Loop Fastener |
| 901 | Hook |
| 902 | Loop |
| 910 | Hook and Loop Fastener Loops |
| 2000 | Screen/User Interface |
| 2001 | Screen/User Interface |
| 2002 | Screen/User Interface |
| 2003 | Screen/User Interface |
| 2004 | Screen/User Interface |
| 2005 | Screen/User Interface |
| 2006 | Screen/User Interface |
| 2007 | Screen/User Interface |
| 2008 | Screen/User Interface |
| 2009 | Screen/User Interface |
| 2010 | Screen/User Interface |
| 2011 | Screen/User Interface |
| 2012 | Screen/User Interface |
| 2013 | Screen/User Interface |
| 2014 | Screen/User Interface |
| 2015 | Screen/User Interface |
| 2016 | Screen/User Interface |
| 2017 | Screen/User Interface |
| 2018 | Screen/User Interface |
| 2019 | Screen/User Interface |
| 2020 | Screen/User Interface |
| 3000 | Control Unit |
| 3001 | Control Unit |
| 3002 | Control Unit |

All measurements disclosed herein are at standard temperature and pressure, at sea level on Earth, unless indicated otherwise. All materials used or intended to be used in a human being are biocompatible, unless indicated otherwise.

It will be understood that each of the elements described above, or two or more together may also find a useful application in other types of methods differing from the type described above. Without further analysis, the foregoing will so fully reveal the gist of the present invention that others can, by applying current knowledge, readily adapt it for various applications without omitting features that, from the standpoint of prior art, fairly constitute essential characteristics of the generic or specific aspects of this invention set forth in the appended claims. The foregoing embodiments are presented by way of example only; the scope of the present invention is to be limited only by the following claims.

1. The exercise mimetic device of claim 2, wherein
the stimulator unit generates and transmits low or high intensity electrical stimulation, is sized for portable use, and includes:
a power source;
a means for generating electrical stimulation;
a wireless connection means;
a plurality of channels for directing electrical stimulation, wherein each channel is associated with the stimulation of a particular muscular group; and
one or more non-transitory computer-readable storage media embodying logic that is operable when executed to:
create, store, and execute programs outlining electrical stimulation parameters;
receive and execute commands via the stimulation unit wireless connection means for generating and transmitting electrical stimulation;
receive commands via the stimulation unit wireless connection means to execute programs; and
send feedback and parameter information via the stimulation unit wireless connection means;
the control unit is sized for portable use and includes:
a user interface;
a power source;
a wireless connection means; and
one or more non-transitory computer-readable storage media embodying logic that is operable when executed to:
receive commands via the interface;
wirelessly connect to the stimulator unit via the control unit wireless connection means;
send commands to stimulator unit via the control unit wireless connection means for generating and transmitting electrical stimulation;
send commands to stimulator unit via the control unit wireless connection means to execute programs;
receive feedback and parameter information from the stimulator unit via the control unit wireless connection means; and
display the feedback and parameter information on the interface; and
the electrodes are positioned on the inner surface of the garment, are in contact with the skin of a wearer when worn, are electrically connected to the stimulator unit, and conducts the electrical stimulation to the wearer.

2. An exercise mimetic device for simulating low or high intensity exercise using low or high intensity electrical stimulation to generate low or high intensity muscle contractions comprising:
a) a stimulator unit;
b) a control unit; and
c) a garment comprising electrodes and an opening allowing for bathroom usage when the device is worn.

3. The exercise mimetic device of claim 2, wherein the garment is made up of upper body and lower body pieces.

4. The exercise mimetic device of claim 2, wherein the garment includes zippers positioned on the legs of a wearer.

5. The exercise mimetic device of claim 2, wherein the garment is made up of a fabric or stretchable material and further comprising:
d) wires electrically connecting the stimulator unit to the electrodes;
e) channels embedded within or attached to the fabric and having a circumference that is greater the circumference of the wires; and
f) wherein the wires are threaded through the channels.

6-8. (canceled)

9. A method for simulating low intensity exercise using the device of claim 2.

10. A method for treating obesity, obesity related conditions, muscle toning, and other conditions benefitted by exercise using the exercise mimetic device of claim 2.

11. A method for increasing muscle tone or producing muscle toning using the exercise mimetic device of claim 2.

12. A method for simulating high intensity exercise or muscle contractions using the exercise mimetic device of claim 2.

13. The exercise mimetic device of claim 1, wherein the control unit includes a means for recharging the control unit power source.

14. The exercise mimetic device of claim 5 further comprising a means for running wires and storing wires and plurality of electrodes, wherein the means is removably affixed to the garment.

15. The exercise mimetic device of claim 1, wherein the stimulator unit further comprises a means for recharging the stimulator unit power source.

16. The exercise mimetic device of claim 2, wherein the garment and the garment is made up of a material having a lower weight, increased absorbency, or improved temperature regulating characteristics than neoprene.

17. An apparatus for simulating exercise comprising a garment worn by a user having an opening in the crotch area of the user and a neuromuscular electrostimulation (LAMES) system for generating muscle contractions that is removably attached to the garment.

18. The apparatus of claim 17, wherein the NMES system includes:
a plurality of electrodes positioned on the inner surface of the garment and in contact with the skin of a wearer when worn;
a stimulator unit for generating electrical stimulation; and
a plurality of wires for connecting and transmitting the electrical stimulation from the stimulator unit to the plurality of electrodes.

19. The apparatus of claim 17, wherein the garment includes an upper section worn on an upper body of the user and a lower section worn on a lower body of the user.

20. The apparatus of claim 19, wherein the NMES system include an upper NMES removably attached to the upper section and a lower NMES system removably attached to the lower section.

21. The apparatus of claim 19, wherein the upper section can be secured to the lower section.

22. The apparatus of claim 19, wherein the upper section is secured to the lower section using hooks.

23. The apparatus of claim 19, wherein the upper section is secured to the lower section using hook-and-loop fasteners.

24-28. (canceled)
